(19) United States
(12) Patent Application Publication
(10) Pub. No.: US 2010/0331580 A1
(43) Pub. Date: Dec. 30, 2010

(54) OIL RECOVERY AND SYNGAS PRODUCTION FROM BIOMASS-BASED PROCESSES

(75) Inventor: William S. Ridgley, Panora, IA (US)

Correspondence Address: MCGUIREWOODS, LLP, 1750 TYSONS BLVD, SUITE 1800, MCLEAN, VA, 22102, US

(73) Assignee: Growmark, Inc., Bloomington, IL (US)

(21) Appl. No.: 12/641,852

(22) Filed: Dec. 18, 2009

Related U.S. Application Data
(63) Continuation-in-part of application No. 11/939,191, filed on Nov. 13, 2007.
(60) Provisional application No. 60/858,960, filed on Nov. 15, 2006.

(51) Int. Cl. C07C 31/08; B01D 11/00; B01D 3/00; B01D 12/00; C01B 3/02

(52) U.S. Cl. 568/840; 210/173; 202/169; 252/373

(57) ABSTRACT

A biomass-based oil extraction process is disclosed. The process includes the recovery of biomass-based oil and other co-products, including but not limited to steam, electric power and chemicals, from various biomass processes and in particular, a process that involves dry biomass milling methods. The process involves extraction of oil from milled biomass-based products and residues from the fermentation step, including thick stillage, distillers wet grain, distillers dry grain and distillers dry grains with solubles, by the application of an alkyl acetate, phase separation and recovery of the separated matter. A process of drying wet co-product using ethanol and carbon dioxide from the production facility is also disclosed. Also a process for the production of syngas from oil containing or deoiled biomass-based products in a pressurized gasifier is disclosed.

### CROSS REFERENCE TO RELATED APPLICATIONS

This application is a continuation in part of U.S. patent application Ser. No. 11/939,191, filed on Nov. 13, 2007, which claims priority to U.S. Provisional Patent Application No. 60/858,960, filed on Nov. 15, 2006, the contents all of which are incorporated herein by reference in their entireties.

### SCOPE OF THE INVENTION

The invention generally relates to biomass-based processes, including corn processes, and more specifically to the extraction of oil and other co-products from biomass, including co-products associated with various biomass processes such as an ethanol production process.

### BACKGROUND OF THE INVENTION

As may be known, biomass process technologies that may include the production of ethanol from biomass, produces various by-products or co-products. Biomass may include any material comprising at least carbon, hydrogen, and oxygen, including corn, sugarcane, hemp, willow, sorghum, wheat, barley, rice, other seeds, various tree species, plant, animal or agricultural waste, other biological material, or the like.

As one example of a product extraction process from biomass, in the production of ethanol after fermentation, continuously pumping beer from fermentors into a still serves to separate the alcohol from various co-products. Typically, in the process, from the distillation column, which often includes 6-16 wt % or greater total solids (typically, 14 wt % of solids are in liquid, either suspended or dissolved), the non-volatile suspended and dissolved solids in the feed are washed down through a lower stripping section and a stream, thick stillage (TS) containing less than 0.02 wt % ethanol may be removed from the bottom of the tower or distillation column. The temperature of this stream exiting the distillation column may be quite high. For instance, even after some heat recovery, the exit temperature of the stream pumped out of the distillation column ranges from 95-99° C. As indicated, this TS exit stream typically contains approximately 14 wt % solids. Two-thirds of these solids generally exist as a suspension; the remainder may be dissolved in liquid. The TS stream may be typically centrifuged and separated into two independent streams, one containing the suspended solids (typically around 35 wt %) and the other stream, thin stillage, containing water and dissolved solids. Each stream may be progressively dried to yield the desired products.

As may be known, the TS may be further processed. For example, the suspended solids stream containing approximately 35 wt % solids called distillers wet grains (DWG), typically has a shelf life of approximately 3 to 5 days and can only be sold to farm operations in the immediate vicinity of an ethanol plant. The stream may be dried to produce distillers modified wet grains (DMWG), containing roughly 50 wt % solids, which typically has a shelf life of about 30 days and can only be sold in regional markets within the region of the ethanol plant. Alternatively, the stream can be further dried to produce distillers dry grains (DDG), having about 90 wt % solids. Typically, at this stage the stream has been dried to roughly 10 wt % or less water and typically has a shelf life of 2-5 years. This product may be sold and shipped throughout the world.

The thin stillage, which includes dissolved solubles in water, may also be further processed. For instance, the thin stillage stream may be dried to produce condensed distillers solubles (CDS), which may include about 35 wt % solids and has a short shelf life. This product may be typically blended with DWG for sale. The thin stillage can be further dried to form modified distillers solubles (MDS), containing roughly 50 wt % solids and typically has a 6 month shelf life when stored in a CO₂ blanket bladder. This product may be typically blended with DMWG for sale. Thin stillage may be further dried to form distillers dried solubles (DDS), containing about 90 wt % solids, which has a one year shelf life. DDS can be sold independently, or can be combined with DDG to form distillers dried grains with solubles (DDGS) for sale.

Each of the foregoing products may be primarily sold as feed. Thus, many of the co-products typically produced from biomass processes such as the ethanol production process, have a limited shelf life and are of limited value and market.

Accordingly, a need exists for a process of efficiently and effectively obtaining additional co-products from biomass processes, such as the ethanol production process both upstream and downstream of fermentation, to improve value gained from biomass technologies that may include corn fermentation technologies, and reduce waste.

### SUMMARY OF THE INVENTION

The invention discloses a process for extracting oil and other co-products from biomass in an ethanol production process, as one example. The process may include obtaining biomass, such as a corn-based biomass, from the ethanol production process, application of an alkyl acetate solvent to the biomass to extract oil so as to produce an extraction solution of at least biomass-based product solids, oil, solvent and water, separating the extraction solution into a first phase containing solvent and oil and a second phase containing at least one of water and solids, separating the first phase from the second phase and removing the solvent from the oil. Application of the alkyl acetate solvent may occur prior to fermentation in the ethanol production process, or post fermentation in the ethanol production process in which it may be applied to at least one byproduct of the fermentation process. The biomass byproducts derived in the ethanol production process may include, but are not limited to, TS, DWG and DDGS. These byproducts may also be derived from other biomass production processes, such as peanut oil extraction, soybean oil extraction, palm oil extraction, oil extraction from other nut meats such as walnut, groundnuts, rapeseed, cottonseed, shea nuts and/or copra oil extraction.

The invention also discloses a process for producing syngas and other products from oil containing or deviled biomass. This process may include obtaining biomass from a biomass harvesting process, reducing the size of the biomass in a particle size reduction process, feeding the reduced biomass into a pressurized gasifier to produce syngas, removing sulfur compounds and moisture from the syngas and applying compression to further remove moisture content in the syngas. The syngas may further be processed to produce, but is not limited to, anhydrous ammonia, dimethyl ether, mixed alcohols, diesel, methanol, butanol, and pure hydrogen.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and the various ways in which it may be practiced. In the drawings:

FIG. 1 illustrates a traditional co-product flow chart of an ethanol production process, post fermentation.

FIG. 2 illustrates a process flow chart, showing the alternate extraction locations utilized in an aspect of the invention.

FIG. 3A illustrates a process flow chart according to one aspect of the invention in which extraction of oil may be from biomass, such as corn-based biomass.

FIG. 3B illustrates a process flow chart of an alternative aspect of an upstream oil extraction process where the extraction entails the use of two separation columns.

FIG. 4A illustrates an alternative process flow chart for a TS stream according to an aspect of the invention.

FIG. 4B illustrates a further manipulation of the TS stream to produce a high protein mash and a low protein mash.

FIG. 5 illustrates a process flow chart according to an alternative aspect of the invention in which extraction of oil may be from TS.

FIG. 6 illustrates a process flow chart of an ethanol-based filter cake drying process according to one aspect of the invention.

FIG. 7 illustrates a process flow chart according to an alternative aspect of the invention in which extraction of oil may be from DWG.

FIG. 8 illustrates a process flow chart according to an alternative aspect of the invention in which extraction may occur at DDGS.

FIG. 9 illustrates a process flow chart of a five-stage re-boiling process according to an aspect of the invention.

FIG. 10 illustrates a process flow chart of energy production from deoiled DWG according to an aspect of the invention.

FIG. 11A illustrates a process flow chart of a biomass synthetic gasification process according to an aspect of the invention.

FIG. 11B illustrates a process flow chart in which anhydrous ammonia may be extracted from synthetic gas by use of an oxygen plant as a source of nitrogen, in an ammonia production process.

FIG. 11C illustrates a process flow chart according to an alternative aspect of the invention in which dimethyl ether may be extracted from synthetic gas by use of at least one step converter.

FIG. 12 is a table illustrating a comparison of alkyl acetate solvents used for extraction at varying temperatures for alternative ethanol production co-products.

FIG. 13 illustrates the temperature profile in a five-stage reboiler desorption unit.

It should be understood that these figures depict aspects of the invention. Variations of these aspects will be apparent to persons skilled in the relevant art(s) based on the teachings contained herein. For example, the flow charts contained in these figures depict particular operational flows. However, the functions and steps contained in these flow charts can be performed in other sequences, as will be apparent to persons skilled in the relevant art(s) based on the teachings contained herein.

### DETAILED DESCRIPTION OF THE INVENTION

The embodiments of the invention and the various features and advantageous details thereof are explained more fully with reference to the non-limiting embodiments and examples that are described and/or illustrated in the accompanying drawings and detailed in the following attached description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention. Moreover, it is noted that like reference numerals represent similar parts throughout the several views of the drawings.

The invention may be generally directed to biomass processes that may include oil extraction. The process includes the recovery of oil and other co-products from biomass, that may include, but are not limited to steam, electric power and chemicals from biomass processes such as ethanol production process(es) and in one example, processes that involve dry corn milling methods. Generally, the process involves extraction of oil from biomass, such as milled corn and/or from residues from the fermentation step, including, but not limited to TS, DWG, DDG, DDGS and the like, and use of a food-grade solvent, such as but not limited to an alkyl acetate. These methods may be applied in existing facilities as retrofits, or may be applied in stand alone new process plant construction such as ethanol plant construction.

To remove oil from still residues, the solvent employed preferably includes an intrinsic hydrophobicity and includes properties making the solvent suitable for environmental, safety and health considerations. The solvent also preferably boils at a temperature favorable for its intended application and in particular, may be acceptable for use in association with the ethanol production process and temperature ranges therein. More specifically, the solvent employed may be capable of use at alternate stages of oil extraction associated with the biomass processes described herein and more preferably between 60° C. and 99° C. and most preferably between 70° C. and 80° C., the maintained temperature of the stillage. The solvent further may include a low solubility in water. Additionally, water also has low solubility in the solvent employed. The preferred solvent used for extraction may be an ester and more preferably, alkyl acetate and most preferably an alkyl acetate azeotrope. Exemplary alkyl acetates suitable for use as an extraction solvent include ethyl acetate, isopropyl acetate and butyl acetate and more preferably, ethyl acetate, although additional acetates and solvent compositions are contemplated. These solvents are currently commercially available from Celanese Corporation (Dallas, Tex.). In the preferred aspect, ethyl acetate forms an azeotrope at approximately 91.8 wt % ethyl acetate and 8.2 wt % water, boiling at 70.4° C. The esterification of acetic acid with ethanol, isopropanol, and butanol produces suitable solvents ethyl acetate, isopropyl acetate and butyl acetate respectively. Alternative solvents having any one or more of the foregoing properties would also be acceptable for the purposes of the invention.

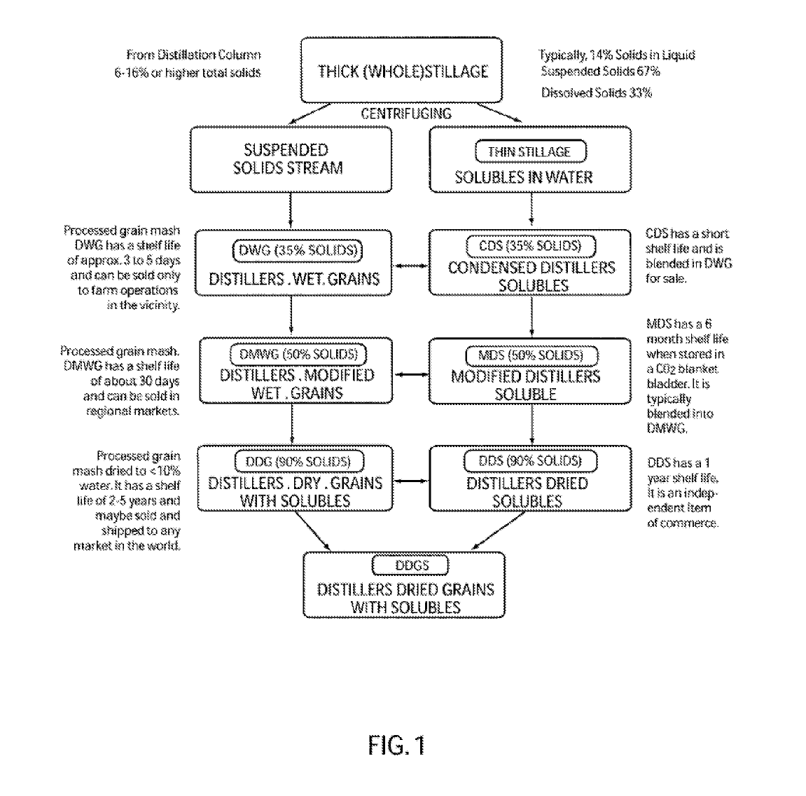

FIG. 1 represents a chart showing a traditional process flow in a biomass process such as the ethanol production process, post fermentation. The process may include the production of common co-products of the ethanol production process, namely, DWG, DMWG, DDG, DDS, DDGS, and the like, all of which can be produced from the TS stream exiting the distillation column of the ethanol production facility. The TS stream from the ethanol production process may be derived from other sources or production processes such as peanut oil extraction, soybean oil extraction, palm oil extraction, oil extraction from other nut meats such as walnut, groundnuts, rapeseed, cottonseed, shea nuts and/or copra oil extraction.

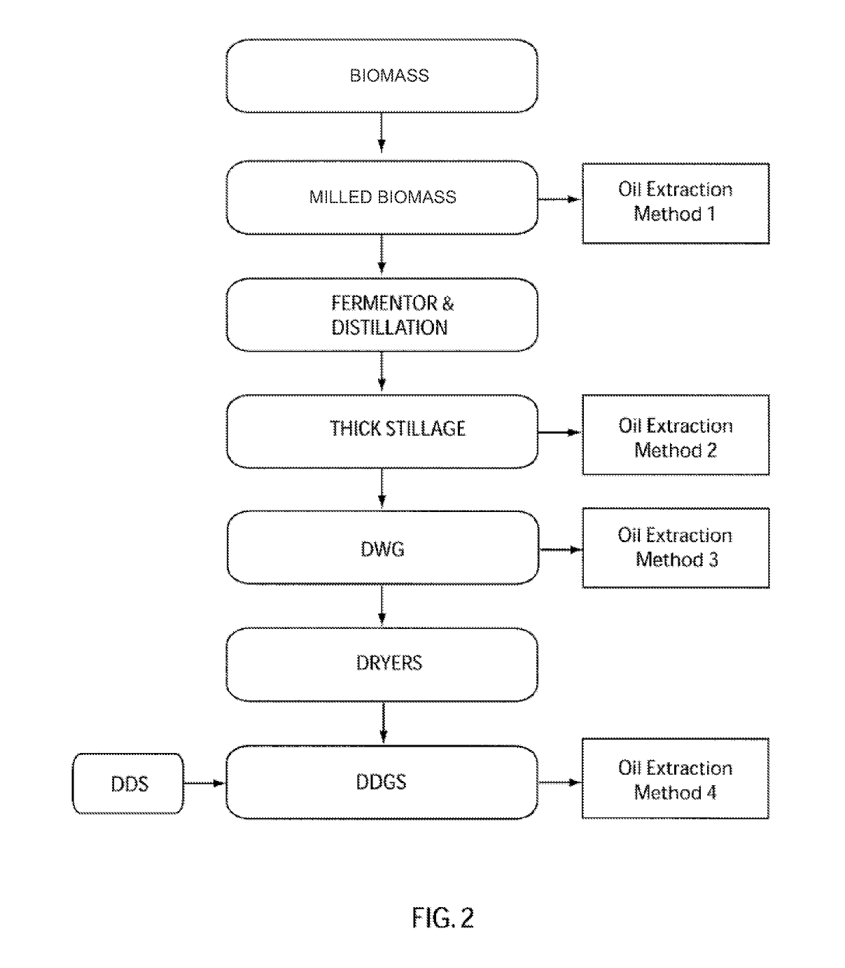

Generally, as shown in FIG. 2, the extraction step may occur in two locations of an ethanol or other biomass-related production process. The process may occur upstream of the fermentors (method 1) or downstream, following fermentation. Application of the extraction step may occur at three alternative stages of the downstream process. In one aspect, the extraction process may be applied to the TS (method 2). In an alternative aspect, the extraction process may be applied to the DWG (method 3). In a further alternative aspect, the extraction process may be applied to the DDGS (method 4). Many of the byproducts produced by one or more of the processes described herein may be recycled into the ethanol production process and biomass oil extraction process. The methods described herein are numbered for purposes of ease of reference only. The methods and numbering thereof are not intended to be arranged in any particular order. One of skill in the art would understand that alternative method numbering, additional methods and/or combinations of the various methods described herein would not depart from the overall scope of the invention.

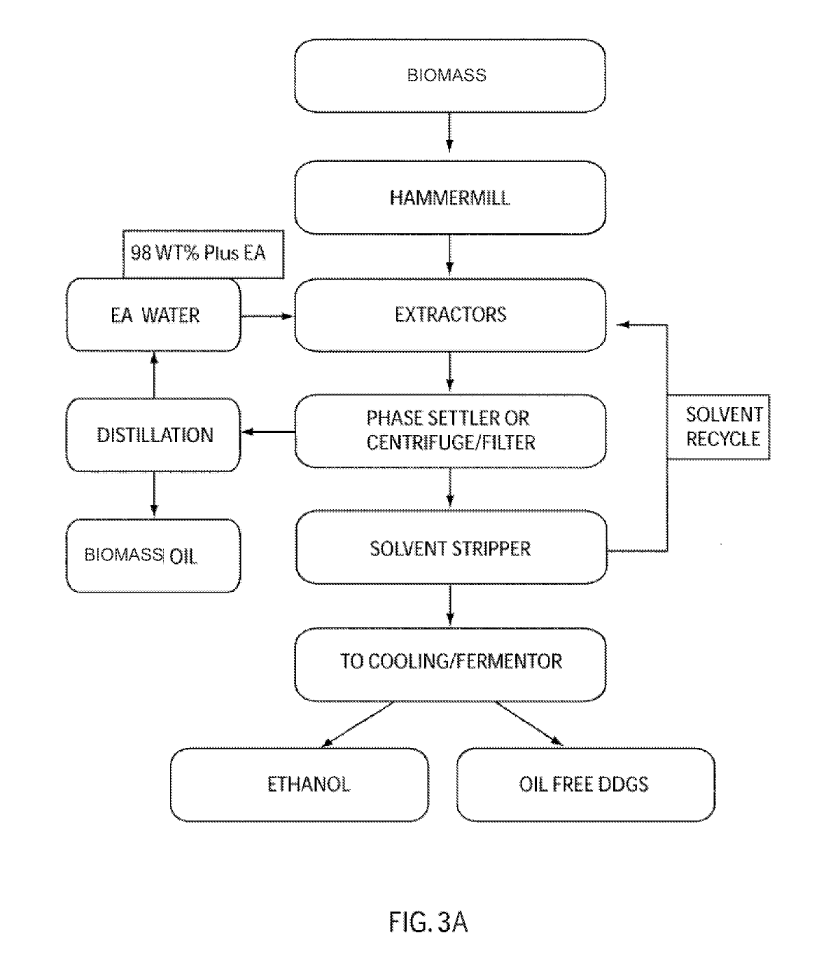

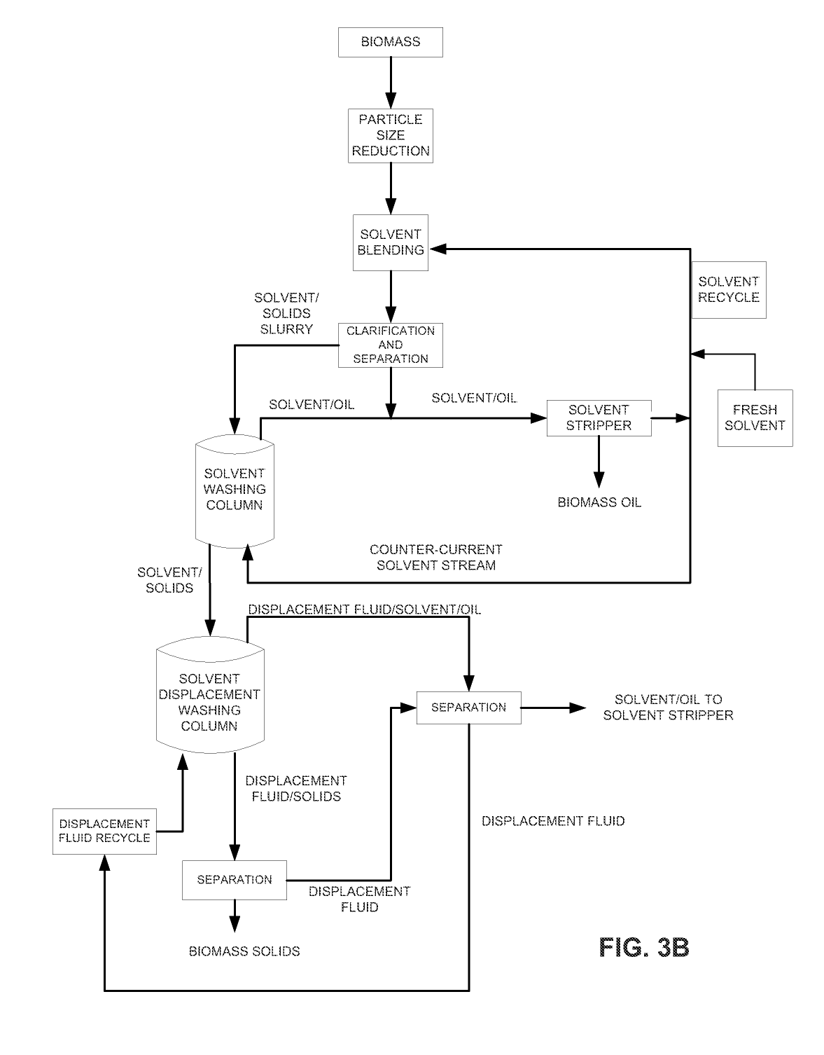

More specifically, in the aspect illustrated in FIGS. 3A and 3B, extraction occurs prior to fermentation in the ethanol or other production process. In other words, in this “front end” approach, oil may be extracted upstream of the fermentors.

In FIG. 3A, in further detail, the unprocessed biomass, such as corn, may be initially transferred from storage silos into a hammermill. The hammermill grinds the biomass to the required particle size, which may be preferably provided having the following size distribution for corn as an example:

| MESH NO. | WT % |
|---|---|
| 16 | 5.44 |
| 20 | 9.99 |
| 30 | 15.74 |
| 40 | 17.35 |
| 60 | 23.42 |
| Bottom | 28.06 |

Alternative particle sizes would not depart from the overall scope of the invention. Following grinding of the biomass, the milled biomass may be transferred to the extractors. Preferably, commercially available conveyor systems linking the extractors with the hammermill transfer the milled biomass. The hammermill and extractors are common commercially available equipment in the ethanol, feed, and food industries or may be custom built for the particular application. In the extractors, solvent may be added or applied to the milled biomass. Preferably, the solvent added may include fresh makeup solvent plus recycled solvent obtained from the process, such as from a solvent stripper, although variations or lack of such blend are also contemplated. The solvent or solvents are intensely blended with the milled biomass to dissolve the biomass oil content into the solvent. In one aspect, the milled biomass may be contacted with the solvent during the transfer process. By the time the milled biomass reaches the extraction area, the solvent and milled biomass have been in contact for a sufficient period of time such that the oils are removed from the milled biomass and extraction may be undertaken without further delay. Generally this transfer process takes from about 2 to about 15 minutes, in particular from about 5 to 10 minutes and in particular about 3 to about 7 minutes. Blending may be conducted in any way commonly practiced in the art. For example, blending may involve use of a stirred tank vessel where intense mixing may be generated by using an agitator. Alternately, blending may be conducted by using centrifugal agitators followed, in some instances, by a static soak tank. Following blending with the solvent and extraction of the oil, a separation step occurs in the phase settler which in one preferred aspect separates the mixture into two separate and discreet phases. The top phase, including the dissolved biomass oil in solvent, may be pumped into a simple distillation column in which the solvent and water are removed, leaving biomass oil as the bottom product. The separation step may also occur either through filtration or through centrifugation. The separation step removes the milled biomass solids from the solvent, oil and water which are in the solvent phase. Acceptable separation systems include belt filters, rotary filters, centrifuges, washing columns and other liquid solid separation equipment. Following separation of the milled biomass solids from the solvent phase mixture, the oil may be separated from the mixture by simple distillation. In a preferred aspect, distillation occurs by using a distillation column. The distillation column may be generally equipped with a number of mass transfer stages with the preferred aspect being either a tray-type or a packed column-type distillation column. The water and solvent are recovered from the top of the column and then recycled back into the process, returning the solvent/water mixture to the extractors.

Preferably, the water in solvent phase exists as an azeotrope. Optionally, the solvent may be dewatered although it may not be necessary. In one aspect, the water in solvent phase, using an alkyl acetate, such as but not limited to ethyl acetate, as solvent, contains 91.8 wt % alkyl acetate and 8.2 wt % water. Water content greater than 8.2 wt % in the solvent phase may be further separated by passing through an additional solvent/water distillation column. The preferred distillation column used incorporates an appropriate number of mass transfer stages, with the stages being either a tray type or a packed column type as indicated herein above.

The deoiled milled biomass solids in the bottom solid phase may be subjected to a desolventizing step to remove solvent absorbed by the milled biomass solids. Preferably, solvent may be removed by a solvent stripper.

In FIG. 3B, in further detail, the unprocessed biomass may be reduced to a preferable particle size by various methods, such as a hammermill, roller mill, cracker mill or by other means. A preferable particle size may be one in which greater than 92% of the particles pass through a 16 mesh sieve, with mesh openings of approximately 1.18 mm. Alternative particle sizes would not depart from the overall scope of the invention. The size reduced biomass may then pass to a solvent blending process, where the solvent added may include fresh makeup solvent plus recycled solvent obtained from the process, such as from a solvent stripper or other solvent/oil separation processes. The solvent may be ethyl acetate, alkyl acetate, alkyl acetate azeotrope or the like. The solvent to particle ratio may be blended to a one-to-one solvent to dry meal blend on a volume basis, or with some forms of biomass, a stronger solvent ratio may be used. For example, in the extraction of soy, canola, or peanut oil, the solvent to biomass ratio may be as high as three-to-one. Similar to FIG. 3A, blending may be conducted in any way commonly practiced in the art. The blended solution then may go through a clarification and separation process where the liquid (the solvent/oil mixture), and the solids (the solvent/solids slurry mixture) may be separated.

The liquid solvent/oil solution from the clarification and separation process may then be sent through a solvent/oil separation process, preferably a solvent stripper, where the solvent may be removed from the biomass oil and recycled back to be reused in the solvent blending process.

The solvent/solids slurry from the clarification and separation process may enter the solvent washing column from the top dropping down through the washing column while a counter-current solvent stream may be sent up from the bottom of the washing column. Such a washing column is commercially available from Koch Modular Process Systems, LLC (Paramus, N.J.). The counter current solvent stream may wash the last of the solvent/oil from the biomass solids and force the solvent/oil mixture to leave from the top of the washing column. The solvent/oil mixture may then be sent through a solvent stripper where the solvent may be removed and recycled, and the biomass oil may be separated, collected, and stored for sale or further processing. The remaining solvent/solids mixture may leave from the bottom of the washing column and may enter a fluid displacement washing column.

The solvent displacement washing column, also commercially available from Koch Modular Process Systems, LLC, is similar to the washing column except that it may use a displacement fluid, such as water or other heavy liquids, rather than a solvent to wash the mixture. Water is the preferred displacement fluid, though other heavy liquids may work just as well. The solvent/solids mixture may enter the fluid displacement washing column from the top dropping down through the washing column while a counter-current displacement fluid stream may be sent up from the bottom of the washing column. The displacement fluid displaces the solvent in the mixture. The liquids (displacement fluid/solvent/oil mixture) coming off the top of the washing column may be sent to a liquid-to-liquid separation process where the displacement fluid may be recycled back to the washing column, and the solvent/oil solution may be sent to a solvent stripper to separate and collect the biomass oil. The solvent may then be recycled back for use in the solvent blending process.

The displaced fluid/solids mixture from the solvent displacement washing column may then be sent to a separation process where the displacement fluid may be recycled back to the washing column, and the biomass solids may now be ready for further processing or sale.

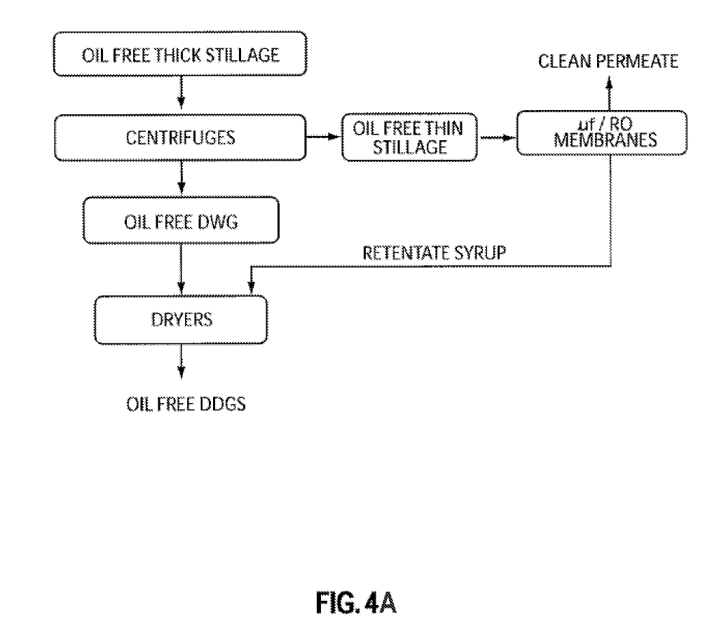

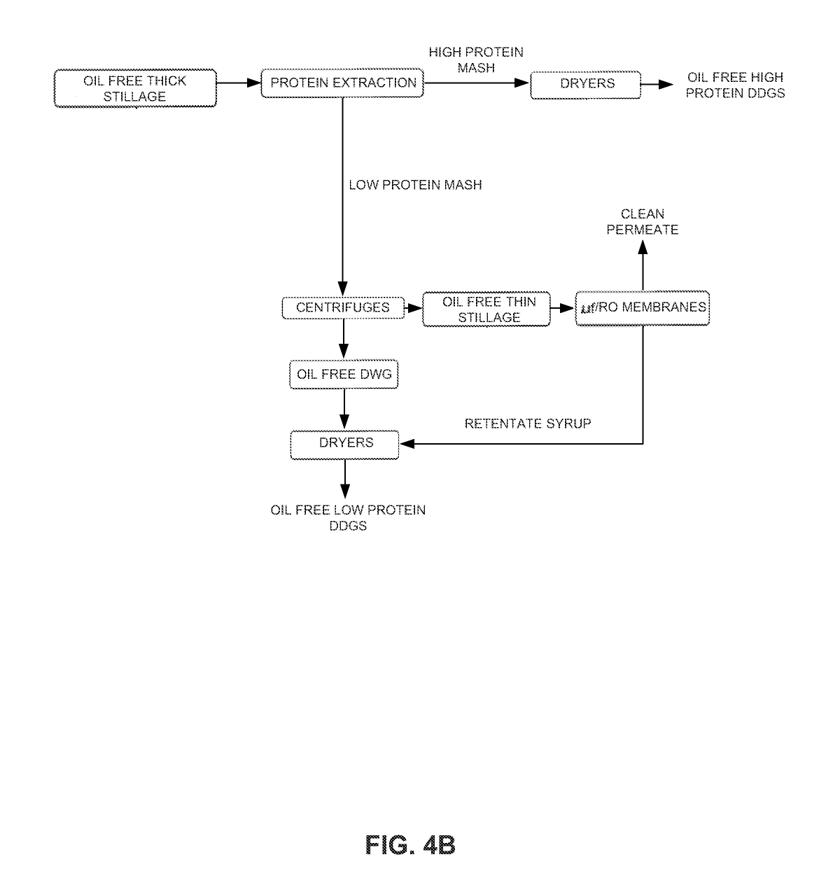

Once solvent is removed, the desolventized milled biomass solids may provide feedstock in one example, for the conventional dry corn milling ethanol production process. This feedstock provides significant advantages, as the downstream process may be simplified and efficiency improved over traditional methods. For example, as illustrated in FIG. 4A, the TS may be placed in a centrifuge to separate the mixture so as to produce oil free DWG and oil free thin stillage. Namely, the TS emanating from the bottoms of the beer column may be oil free and may be passed through the centrifuges where the oil free DWG may be routed to the dryers to dry. The oil free thin stillage may be filtered using a membrane, such as, but not limited to a filtration membrane, including, but not limited to a micro-filtration membrane and/or reverse osmosis membrane. Acceptable membranes are available from Koch Industries (Wichita, Kans.), Siemens Corporation (New York, N.Y.), or GE Osmonics (Minnetonka, Minn.). Filtration results in a clean permeate water stream and retentate syrup. The retentate stream from this operation may be a concentrate of proteinaceous and bacterial matter and may be directed into the dryers for co-blending with DWG as feed to yield an oil free DDGS product. Drying in a dryer may include application of steam and/or direct heated carbon dioxide and preferably, indirect application thereof, to produce oil free DDGS. Drying oil free DWG uses approximately 21% less energy than drying oil containing DWG because it may be easier to dry water rather than oil and water out of a mash.

The TS stream may be further manipulated by a protein extraction process prior to passing the stream through the centrifuge to separate the mixture of oil free DWG and oil free thin stillage. As illustrated in FIG. 4B, a protein extraction process, such as, but not limited to, multiple stages of water wash, may separate the high protein mash from the low protein mash. The high protein mash, which may include over 50% wt crude protein, may be sent to a dryer, dried to approximately 10% moisture before the oil free high protein DDGS may then be sent to storage and sold to the food and feed industry, for example, to be used as food-based additives. The low protein mash, which may include up to 50% wt crude protein, may be sent through the same process as in FIG. 4A to be dried to approximately 10% moisture oil free low protein DDGS. The low protein DDGS may be sold to the feed industry, for example, to be used as live-stock feed.

The thermal energy usage for a commercial dry milled biomass process plant, such as a corn ethanol plant, may be approximately 34,000 Btu/denatured gallon of ethanol produced. In comparison, the ethanol plant utilizing the foregoing process significantly reduces the thermal energy requirements to approximately 23,000 to 24,000 Btu and more preferably, to approximately 23,060 Btu/denatured gallon of ethanol produced. In addition, the pre-fermentation removal of oil reduces fermentation time, saving time and energy. Specifically, by removal of oil from the milled biomass product, the enzymes operate more efficiently by working only on the remaining product.

In an alternative aspect of the downstream, or post fermentation, method of extraction (method 2), oil may be removed from the non-volatile residues that are pumped out and preferably continuously pumped out of the still. As discussed, the spent residue from the post fermentation distillation process may be commonly referred to as TS and includes several major co-products. Typically, the TS contains between 10 wt % and 20 wt % solids and more preferably, about 14 wt % solids, both soluble and insoluble. This stream typically exits the still at a temperature ranging from 80° C. to 100° C., or more preferably, between 85° C. and 95° C. and most preferably, a range of 90° C. to 95° C.

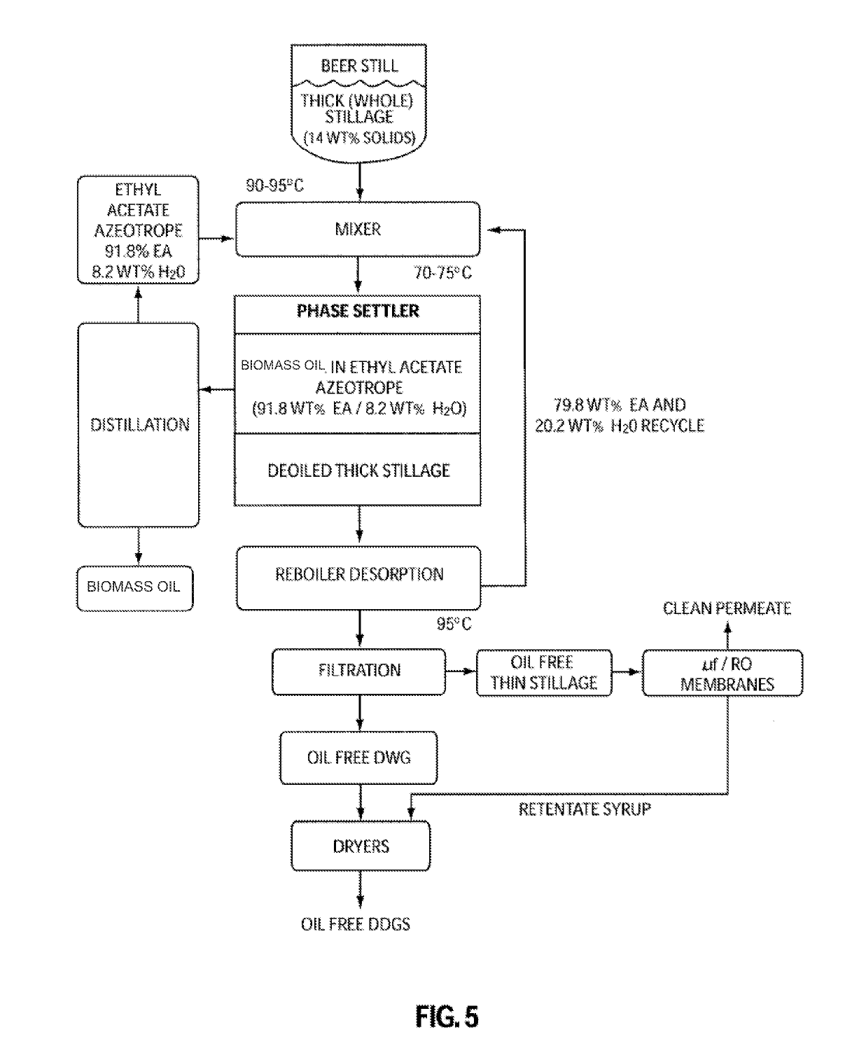

In the method shown in FIG. 5, biomass oil may be extracted from the TS produced downstream of a biomass process facility such as an ethanol production facility. In one preferred aspect, biomass oil, such as corn oil, may be extracted from the TS at the bottoms of the beer still. This also reduces the thermal requirement from 34,000 Btu to 23,060 Btu/denatured gallon of ethanol produced.

Typically, TS in the still typically exists at a temperature of 90° C. to 95° C. TS may be cooled to a temperature of between 60° C. and 80° C. and preferably to a temperature between 65° C. and 75° C., and most preferably, a temperature range of from 70° C. to 75° C. The cooled TS may be blended in a mixer, such as, but not limited to, a conventional stirred tank unit designed to afford an appropriate hold time and/or a centrifugal pump around loop, with a solvent and preferably, an alkyl acetate solvent and more preferably, an alkyl acetate azeotrope. Preferably, the solvent may include a mixture of an alkyl acetate solvent stream, an alkyl acetate azeotrope mixture from a recycle stream of the biomass process such as an ethanol production process (for instance, an ethyl acetate azeotrope containing approximately 91.8 wt % ethyl acetate and 8.2 wt % water) and a recycle stream recovered from a reboiler (containing approximately 79.8 wt % ethyl acetate and 20.2 wt % water). The recycle stream from the reboiler may include a smaller stream than that received from the azeotrope recycle stream. The combined streams may be intensely blended with the TS to dissolve the oil into the solvent.

The blended stream, including the solvent mixture and the TS, may be pumped into a phase settler. In the phase settler the liquids may split into two separate and discrete phases. A preferred phase settler may be a common horizontally configured unit with an appropriate residence time. In some instances, the residence time may be decreased significantly by using electrostatic devices such as those available from NATCO (Houston, Tex.). The phase settler may separate the biomass oil in solvent from the deoiled TS, which separates or settles to the bottom of the phase settler. Namely, as a result of the hydrophobic nature of the alkyl acetate solvent, the liquids separate into two separate and discrete phases. In the preferred aspect, the oil remains in the solvent phase, while protein remains in the water-solid phase. Thus, the top phase may include dissolved biomass oil n solvent. The bottom phase may include deoiled TS in water-solid phase. The top phase may be pumped into a distillation column and preferably, a simple elementary distillation column, where solvent may be removed, leaving biomass oil as the bottoms product. More specifically, the solvent, including alkyl acetate azeotrope, may be boiled out of the solution in the distillation column, leaving corn oil as the resulting product. An acceptable distillation column may be one with an appropriate number of mass transfer stages which may range from two (2) through twenty (20) and include either trays or packing available from Koch-Glitsch (related to Koch Industries of Wichita, Kans.). The number of mass transfer stages may be contingent upon maintaining the desired purity of the recycle-stream.

The lower phase in the settler unit may include primarily water and solids with a small amount of ethyl acetate. The solids included in the lower phase may include both suspended and dissolved solids from the original TS. The lower phase may be decanted into a reboiler desorption unit in which the solvent may be stripped out and recycled back into the mixer. An acceptable reboiler desorption unit may be a stripping column, typically with disc and donut trays offered by Koch-Glitsch (related to Koch Industries of Wichita, Kans.). Preferably, the lower phase mixture may be heated to a temperature of between 70° C. and 110° C. and more preferably, between 80° C. and 105° C., preferably, to a range of from 90° C. to 100° C. and most preferably approximately 99° C. At this temperature, solvent, or more preferably, the alkyl acetate, may be desorbed as a mixture of alkyl acetate and water, forming a mixture that may include between 70 wt % and 90 wt % alkyl acetate and between 10 wt % and 30 wt % water and more preferably, between 75 wt % and 85 wt % alkyl acetate and 15 wt and 25 wt % water and most preferably, approximately 79.8 wt % alkyl acetate and 20.2 wt % water. The desorbed mixture or stream may be preferably recycled back into the mixer and applied to the TS as described hereinabove. In the preferred aspect, the remaining stream of water from the desorption process may contain a minimal amount of solvent and preferably, less than 10 parts per million (ppm) of solvent, keeping solvent loss to a minimum.

The deoiled TS stream resulting from the foregoing process may be concentrated using a dewatering system, such as a filter press, rotary drum, belt, plate and frame, rotary press or other commercially available devices. A suitable dewatering device may be a belt type filtration unit available from Larox Corporation (Lappeenranta, Finland). In a preferred aspect, the dewatering device may be capable of optimization so as to yield a stream of solids which may be approximately 35 wt % solids, having a remainder of water, protein and dissolved solids. Application of the dewatering device to the deoiled TS results in a deoiled filtrate (oil free thin stillage).

The deoiled filtrate may be further cleaned by passing through a membrane. An acceptable membrane includes units that may be available from Koch, Siemens or GE Osmonics described hereinabove. Membranes may be selected in some aspects which are suitable for various water needs, such as a pure water filter arrangement or in which water may be needed or recycled back into the process. In the preferred aspect, the water that passes through the filter may be directed into a reverse osmosis unit. The clean water that exits the filter may be recycled back into the fermentors as described above and the retentate, which may be oil free backset, may be directed to a blending unit, for blending with the milled corn and introduction into a dryer. The retentate stream may be preferably minimal and may be concentrated to yield a protein rich syrup or broth. A wet filter cake of solids may also result from the filtration process. The wet filter cake may typically include residual moisture and often times may include a significant amount of moisture. For example, a wet filter cake may contain between 60-70 wt % moisture.

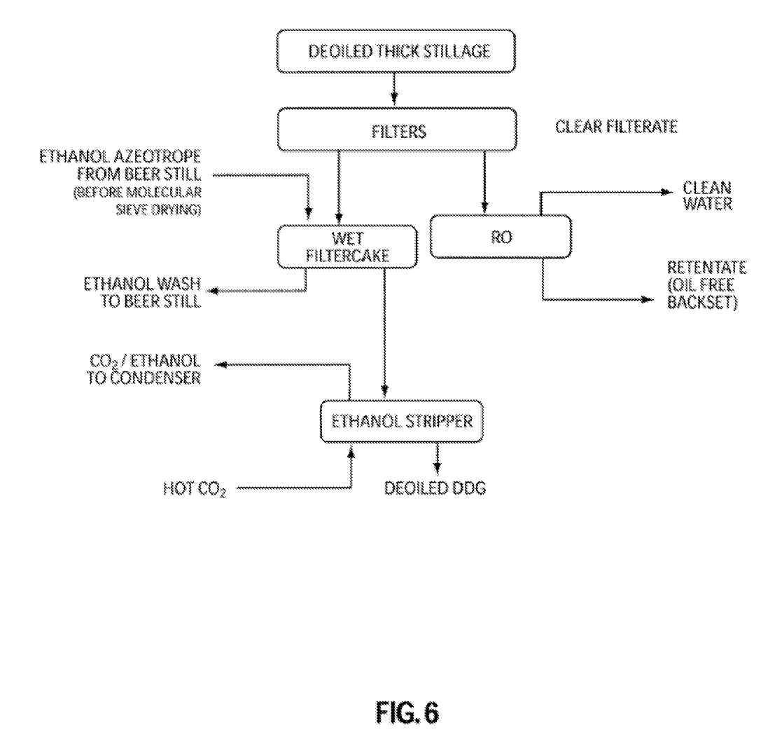

In one aspect, as illustrated in FIG. 6, ethanol from the production process and more preferably, ethanol azeotrope formed from the overheads of the beer still may be used to wash the wet filter cake. The composition of solution used to wash the wet filter cake may include between 80 wt % and 100 wt % ethanol and more preferably, between 90 wt % and 100 wt % ethanol and most preferably approximately 95 wt % ethanol. This ethanol wash solution may be preferably obtained from the still prior to molecular sieve drying. The ethanol laden wash stream may be washed over the filter cake and dissolves the moisture remaining in the wet filter cake, resulting in a filter cake loaded with ethanol and a minimal amount of residual moisture, or water. The wet ethanol wash stream containing the moisture or water from the filter cake may be redirected into the beer still for separation. Thus, in the preferred aspect, the moisture in the filter cake dissolves into the ethanol leaving the filter cake ethanol rich.

The ethanol rich filter cake may then be dried. In the preferred aspect, the cake may be dried by the application of a stream of a gas having inert characteristics. CO₂ may be a fermentation byproduct and may be a readily available stream having inert characteristics. Preferably, the stream of inert gas may be heated. Accordingly, in one preferred aspect the process uses a heated stream of CO₂, which may be obtained from the fermentation process by recovering this stream downstream of the deodorant adsorbers. Preferably, the CO₂ stream may be recovered by utilizing a pressurizing device which may be a recycle compressor/fan to move the CO₂ through dryers. The stream may be preferably heated to a temperature above 70° C. and below 120° C. The stream preferably has a concentration of CO₂ ranging from 80 mole % to 100 mole %, which stream may include a diluent, such as water vapor and in some instances, a small amount of air which has occluded into the stream. The heated stream of CO₂ or inert gas applied to the ethanol rich filter cake desorbs the ethanol from the filter cake, yielding a deoiled filter cake or DDG product and a CO₂/ethanol stream. The ethanol/CO₂ stream may be routed to a conventional condenser in which the ethanol may be removed as a liquid and the CO₂ recycled.

In another aspect, a solvent may be used to wash and dewater the wet filter cake. In a preferred aspect, the solvent is alkyl acetate or alkyl acetate azeotrope. The alkyl acetate or alkyl acetate azeotrope laden wash stream may be washed in a counter current over the filter cake to dissolve the moisture remaining in the wet filter cake, resulting in a filter cake loaded with alkyl acetate or alkyl acetate azeotrope, with a minimal amount of residual water. The acetate rich filter cake may then be dried, in one preferred aspect, in the same manner as the ethanol rich filter cake.

The filter cake from the presses or filtration system may be alternatively dewatered using conventional mechanical/thermal processing, such as, but not limited to, passing it through a rotary drum dryer, which may be directly or indirectly fired based upon energy optimization. In a preferred aspect, conventional means may be applied to yield a stream containing about 70 wt % solids and 30 wt % water. The filter cake from the presses may be suitably dewatered by conventional means to yield an oil free DDG. Alternatively, the output stream may be optimized for feeding into a pressurized gasifier for the production of synthetic gas (i.e., syngas). Optimization may include the consideration of atmospheric or pressurized dewatering. Often times, the stream may include a rich, heavy slurry. In this instance, it may be appropriate to use concrete pumps, such as a Putzmeister for pumping the stream. Gasifiers suitable for use include moving grate-types of units available from KMW Systems, Inc. (London, Ontario) and pressurized units like the type available from Carbona, Inc. (Helsinki, Finland). In some instances, a continuous gasification process will occur which may not need any lock hoppers and/or corresponding equipment. Syngas, as may be known, which may include almost pure hydrogen, may be further processed by known methods for a variety of purposes, including, but not limited to the production of anhydrous ammonia, ethanol, dimethyl ether, and other alcohols and biofuels, or the like.

The biomass oil generated using the foregoing method may be acceptable for use in biodiesel production, or may be sent to a refining facility for additional product handling or refining, to sell the oil as a food grade material. Additionally, the ethanol-drying methodology described provides significant advantages, as it may eliminate conventional drying methodology and its incumbent heavy capital costs, energy costs and emission concerns. Moreover, the absence of oil in the filter cake eliminates the plugging problems of traditional systems that otherwise prevent use of reverse osmosis or micro-filtration membranes. This downstream process also appears to have the same capability as the first approach of reducing the thermal requirement of the ethanol facility to 23,060 Btu/denatured gallon of ethanol produced.

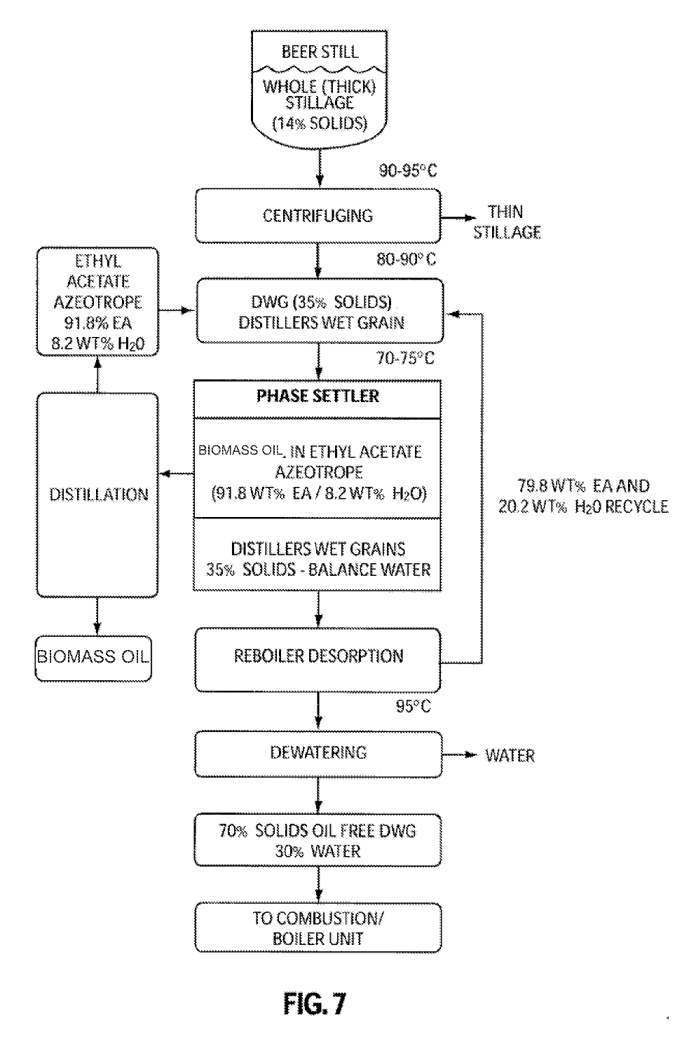

In method 3, or the second downstream method of biomass oil extraction, shown in FIG. 7, oil may be extracted from the DWG produced downstream of the biomass facility such as an ethanol production facility. In this aspect, the TS may be passed through a centrifugal liquid-solid separation device and may be split into thin stillage and DWG. Suitable separation devices include centrifuges that may be available from Flottweg AG (Vilsbiburg, Germany) and Westfalia Technologies, Inc. (York, Pa.). The separated DWG may include insoluble solids and water and more specifically a proportionate amount of water. Typically, the DWG stream may include 35 wt % solids in water. Centrifugation results in a temperature reduction of the DWG. Preferably, the temperature drops to a range of between 75° C. and 95° C. and more preferably a range of 80° C. to 90° C. The DWG stream may be blended with solvent and preferably a three stream solvent similar to that described with the prior aspect, including an alkyl acetate, such as ethyl acetate, an alkyl acetate azeotrope recycle stream (preferably having a concentration of 91.8 wt % ethyl acetate and 8.2 wt % water), plus a small recycle stream from the reboiler (preferably having a concentration of 79.8 wt % ethyl acetate and 20.2 wt % water). The small recycle stream may be available from the recovery of the alkyl acetate which may be dissolved in the process in a large quantity of water. The streams may be intensely blended with DWG in a mixer to dissolve the oil in the solvent.

As with the previously described aspect, the solvent stream having dissolved biomass oil may be pumped into a phase settler, which separates the stream into two separate and discreet phases. The top phase includes the dissolved oil in solvent. The top phase may be, as discussed above, pumped into a simple distillation column in which the solvent may be removed, leaving biomass oil as the bottoms product. The lower phase in the settler, which may include solids in water, may be as previously described, decanted into a reboiler desorption unit in which any remaining solvent may be stripped out and recycled back into the mixer as described above.

The remaining DWG stream, free of biomass oil, may be dewatered as previously described or may be dewatered to 30 wt % water which may be ideal for feeding into a pressurized gasifier for the production of syngas as was described for the TS above to produce the deoiled DWG having a moisture level of 30 wt %. The thermal energy requirement slightly reducing from 34,000 to 32,820 Btu/denatured gallon of ethanol.

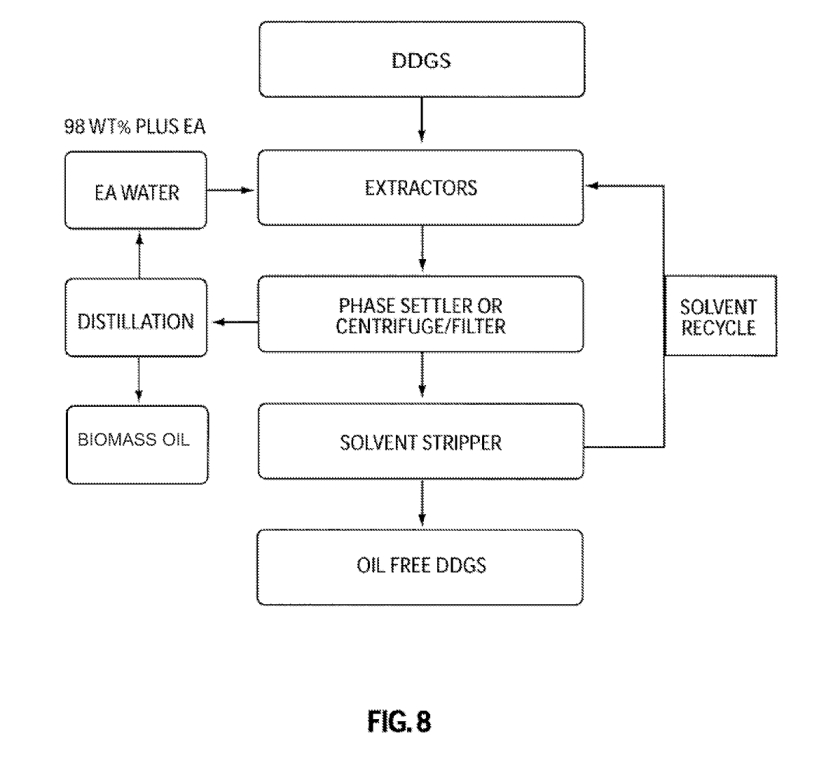

In method 4, or the third downstream method of biomass oil extraction, oil may be extracted from the final byproduct of the biomass process such as an ethanol production process or drying process, namely DDGS, the dry solid residue. As illustrated in FIG. 8, similar to the biomass oil extraction from milled biomass using an alkyl acetate solvent, the DOGS may be intensely blended with the solvent to dissolve the biomass oil content in the DDGS into the solvent. The oil may then be extracted in the same way as previously described for the biomass oil extraction from milled biomass (method 1).

The foregoing processes provide for significant improvement in the productivity of a biomass process plant such as an ethanol production plant and more specifically may increase productivity by nearly 20 wt %. In addition, for each of the foregoing processes, the product remaining in each process, after the oil has been extracted, consists primarily of cellulosic and proteinaceous components and the like that may be sold as animal feed. This deoiled material and residue also has value as fuel and may be used to raise steam and/or generate power, including but not limited to power for the production facility or other facilities. As one example, for deoiled DDGS, namely, one (1) bushel of corn (56 lbs.) yields 16.8 lbs of deoiled DDGS having a fuel value of 6,900 Btu/lb and corn oil in DDGS having a fuel value of 1,500 Btu/lb. In addition, food grade oil may be extracted using one or more of the foregoing methods. This oil can then be sold for food applications. Alternatively, the oil may be used as a feed stock for producing biodiesel. In addition, the deoiled milled biomass and residues provide feedstock for the conventional dry corn milling ethanol plant where low energy filters are used in place of evaporators. The deoiled residues may also be gasified to produce syngas for production of ethanol, methanol, other chemicals, dimethyl ether (DME) and other power or fuel applications, or the like, and they can also be pelletized for consumption as animal feed.

### EXAMPLES

The following Examples, are directed to biomass generally though the specific examples are of corn biomass, are set forth so as to provide those of ordinary skill in the art with a complete disclosure and description of the methods claimed herein, their performance and evaluation and are intended to be purely exemplary of the invention and are not intended to limit the scope of what may be regarded as the invention. Efforts have been made to ensure accuracy with respect to numbers (e.g., amounts, temperature, etc.) but some errors and deviations should be taken into account.

### Example 1

### Oil Extraction From TS or DWG

The following analysis was performed related to oil extraction from TS or DWG.

TS and DWG samples were acquired from one corn-based biomass dry mill ethanol plant nearby and stored at −80° C. prior to usage.

Extraction of corn-based biomass oil from TS or DWG was carried out using the assigned solvent and temperature based on the experimental designs. Triplicate extractions were conducted for each experimental condition. For each extraction, 25 mL of solvents were mixed with 2-10 g of TS or DWG. The extraction was conducted using a shaker for 1 hour, with a water bath to control the temperature. For each experiment, after the organic phase was separated, the residue was extracted for the second time using 15 mL of the same solvent for 30 minutes. The organic phases were combined for oil analysis. The solid residue was dried.

The corn-based biomass oil content in TS or DWG was analyzed using AOAC (Association of Official Analytical Chemists) Official Method 945.16 (Petroleum Ether Extraction Method). The corn-based biomass oil was extracted from TS or DWG with petroleum ether for 6 hours. The extract was filtered through small, hardened paper into a weighed vessel and then the paper was washed with a small portion of hot fresh solvent. After the solvent was evaporated at temperature of 70° C. and the dry vessel containing residue was dried in air in an oven for 1 hour at 100° -105° C., the weight of the corn-based biomass oil extracted was measured using a balance.

The content of water in TS or DWG was analyzed using AOAC Official Method 945.15 (Air Oven Method). The content of water in the solvent phase was analyzed following the method of Karl Fischer titration using a HYDRANAL moisture test kit purchased from Sigma. Gas chromatography coupled with flame ionization detection (GC-FID) was utilized for the analysis of the residue solvents after extraction. An Agilent 6890 gas chromatograph and a J&W Scientific 30-meter-long narrow-bore capillary column (DB5) with 0.25-μm phase thickness were utilized. The method applied for protein content analysis was Onishi & Barr Modified Lowry procedures using a test kit (Sigma TP 0200) purchased from Sigma.

Using ethyl acetate as the solvent, corn-based biomass oil extraction experiments were conducted at three different temperatures: 35° C., 45° C. and 55° C. After extraction, the leftover solids were dried in an oven. Part of the solids was extracted again using petroleum ether to analyze the leftover oil and the other part was used for the analysis of protein content.

Since it may be difficult to obtain a water phase in these experiments, the content of ethyl acetate in water was analyzed in this way: 50 mL of liquid was separated from TS through centrifugation and then the liquid was mixed with the same amount of ethyl acetate. Water phase samples were collected at three different temperatures: 35° C., 45° C. and 55° C. and analyzed using Gas Chromatography.

Using isopropyl acetate as the solvent, corn biomass oil extraction experiments were conducted at six different temperatures: 45° C., 55° C., 65° C., 70° C. 80° C. and 90° C. After extraction, the leftover solids were dried in an oven. Part of the solids was extracted again using petroleum ether to analyze leftover oil and the other part was used for the analysis of protein content.

Since it may be difficult to obtain a water phase in these experiments, the content of isopropyl acetate in water was analyzed in this way: 50 mL of liquid was separated from TS through centrifugation and then the liquid was mixed with the same amount of isopropyl acetate. Water phase samples were collected at six different temperatures: 45° C., 55° C., 65° C., 70° C., 80° C. and 90° C. and analyzed using Gas Chromatography.

In this example, TS and DWG samples were acquired from one corn-based biomass dry mill ethanol plant. Similar results are expected through the use of TS and DWG samples acquired from other types of biomass materials.

### Example 2

### Oil Extraction From TS Using Ethyl Acetate

The following represents one method of extracting oil from TS using ethyl acetate solvent. A total of 9 bench-scale tests were conducted at three different temperatures using ethyl acetate as the solvent. TS was intensely blended with solvent to perform the oil extraction. Experimentation and analysis was performed as set forth in Example 1. The results are shown in Tables 1, 2 and 3.

TABLE 1 summarizes the results of corn-based biomass oil extraction from TS using ethyl acetate as solvent at 35° C. At 35° C., the Specific Gravity of ethyl acetate may be 0.8848 and the content of ethyl acetate in water phase may be 45.15 g/L. The amount of TS used in these tests may be normalized to 10 g for comparison purposes.

TABLE 1
Results of Oil Extraction from TS using Ethyl Acetate as Solvent at 35° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| TS1 | 8.6963 | 0.1537 | 1.2505 | 24.59 | 0.12 | 27.5 |
| TS2 | 9.4857 | 0.1611 | 1.3498 | 25.02 | N/A | 27.8 |
| TS3 | 10.7799 | 0.1760 | 1.5437 | 24.73 | N/A | 27.1 |
| Average | 9.6540 | 0.1636 | 1.3814 | 24.78 | 0.12 | 27.5 |
| Normalized | 10.0000 | 0.1695 | 1.4308 | 24.78 | 0.12 | 27.5 |
| to 10 g | | | | | | |

Normalized for 10.0000 g of TS which gives water content of 8.5692 g and dry matter (DM) content of 1.4308 g. The DM may be further divided into oil of 0.0014+0.1695=0.1709 g, protein of 0.3935+0.0049=0.3984 g and other DM of 0.8615 g. The combined extraction solution was allowed to settle into two phases. The solvent phase includes 41.05 ml or 36.3195 g ethyl acetate, 24.78 g/L×41.05 ml=1,0172 g water, 0.12 g/L×41.05 ml=0.0049 g protein and 0.1695 g oil, for a total of 37.5111 g, or 1.1916 g on an ethyl acetate free basis. The Water-solid phase includes water equal to 8.5692−1.0172=7.5520 g, oil equal to 0.0014 g, protein equal to 27.5 wt %×1.4308=0.3935 g, other DM equal to 0.8615 g and ethyl acetate equal to 45.15 g/L×7.5520 ml=0.3410 g=0.3854 ml, for a total of 9.1494 g, or 1.5974 g on a water free basis.

TABLE 2 summarizes the results of corn-based biomass oil extraction from TS using ethyl acetate as solvent at a higher temperature, namely 45° C. At 45° C., the Specific Gravity of ethyl acetate may be 0.8733 and the content of ethyl acetate in water phase may be 48.21 g/L. The amount of TS used in these tests may be normalized to 10 g for comparison purposes.

TABLE 2
Results of Oil Extraction from TS using Ethyl Acetate as Solvent at 45° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| TS1 | 6.5279 | 0.1110 | 0.9387 | 26.21 | 0.12 | 27.7 |
| TS2 | 7.6276 | 0.1369 | 1.0854 | 26.14 | N/A | 27.2 |
| TS3 | 9.3904 | 0.1516 | 1.3447 | 26.16 | N/A | 27.3 |
| Average | 7.8486 | 0.1332 | 1.1229 | 26.17 | 0.12 | 27.4 |
| Normalized | 10.0000 | 0.1697 | 1.4307 | 26.17 | 0.12 | 27.4 |
| to 10 g | | | | | | |

Normalized for 10.0000 g of TS which gives water content of 8.5693 g and DM content of 1.4307 g. The DM may be further divided into oil of 0.0014+0.1697=0.1711 g, protein of 0.3920+0.0061=0.3981 g and other DM of 0.8615 g. The combined extraction solution was allowed to settle into two phases. The solvent phase includes 50.56 ml or 44.1540 g ethyl acetate, 26.17 g/L×50.56 ml=1.3232 g water, 0.12 g/L×50.56 ml =0.0061 g protein and 0.1697 g oil, for a total of 45.6530 g, or 1.4990 g on an ethyl acetate free basis. The water-solid phase includes water equal to 8.5693−1.3232=7.2461 g, oil equal to 0.0014 g, protein equal to 27.4 wt %×1.4307=0.3920 g, other DM equal to 0.8615 g and ethyl acetate equal to 4821 g/L×7.2461 ml=0.3493 g=0.4000 ml, for a total of 8.8503 g, or 1.6042 g on a water free basis.

TABLE 3 summarizes the results of corn-based biomass oil extraction from TS using ethyl acetate as solvent at a higher temperature, namely 55° C. At 55° C., the Specific Gravity of ethyl acetate may be 0.8613 and the content of ethyl acetate in water phase may be 51.18 g/L. The amount of TS used in these tests may be normalized to 10 g for comparison purposes.

TABLE 3
Results of Oil Extraction from TS using Ethyl Acetate as Solvent at 55° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| TS1 | 8.6939 | 0.1522 | 1.2502 | 27.08 | 0.13 | 27.5 |
| TS2 | 8.4482 | 0.1354 | 1.2022 | 26.89 | N/A | 27.2 |
| TS3 | 9.4122 | 0.1680 | 1.3478 | 27.11 | N/A | 27.0 |
| Average | 8.8514 | 0.1519 | 1.2667 | 27.03 | 0.13 | 27.2 |
| Normalized | 10.0000 | 0.1716 | 1.4311 | 27.03 | 0.13 | 27.2 |
| to 10 g | | | | | | |

Normalized for 10.0000 g of TS which gives water content of 8.5689 g and DM content of 1.4311 g. The DM may be further divided into oil of 0.0014+0.1716=0.1730 g, protein of 0.3893+0.0058=0.3993 g and other DM of 0.8588 g. The combined extraction solution was allowed to settle into two phases. The solvent phase includes 44.75 ml or 38.5437 g ethyl acetate, 27.03 g/L×44.75 ml=1.2096 g water, 0.13 g/L×44.75 ml=0.0058 g protein and 0.1716 g oil, for a total of 39.9307 g, or 1.3870 g on an ethyl acetate free basis. The water-solid phase includes water equal to 8.5689−1.2096=7.3593 g, oil equal to 0.0014 g, protein equal to 27.2 wt %×1.4311=0.3893 g, other DM equal to 0.8588 g and ethyl acetate equal to 51.18 g/L×7.3593 ml=0.3766 g=0.4372 ml, for a total of 8.9854 g, or 1.6261 g on a water free basis.

The foregoing results demonstrate that better than 99 wt % of the corn-based biomass oil can be extracted from TS at varying temperatures. Secondly, the extraction results in a majority, or not all, the oil in the solvent phase and protein remaining in the water-solid phase. Third, temperature changes show limited or no effect on the strength of ethyl acetate extraction capability.

In this example, the TS sample was acquired from one corn-based biomass dry mill ethanol plant. Similar results are expected through the use of TS samples acquired from other types of biomass materials.

### Example 3

### Filtration of Two Phase Stillage

As indicated, the extraction solution separates into two phases. A total of 8 lab-scale filtration tests were conducted using a plate and frame type filter press, to filter the solids. A 70 wt % rubbing alcohol wash was used to wash the filtrate. TABLE 4 summarizes the filtration results for filtration tests 3-6 as examples. Experimentation and analysis was performed as set forth in Example 1.

TABLE 4
Summary of Filtration Test #3 Through #6

| Filtration Capacity | 4.0 to 9.2/kg/DS/m²/h |
|---|---|
| Liquid Capacity | 194-401 l/m²/h |
| Cake Moisture | 65.1 |

Some general observations were obtained from the filtrations tests. Specifically, moisture levels of 65 wt % were achieved while using 70 wt % rubbing alcohol as a wash, such results will likely be improved using 100 wt % alcohol/ethanol. Secondly, two phase separation occurs in Mother Liquor. Third, the resulting filtrate may be clear. It may be noted that additional tests on a full scale test unit will likely yield improved results, due to various factors such as dual sided filtration and washing variables available on the particular unit employed.

### Example 4

### Oil Extraction From DWG Using Ethyl Acetate

The following represents one method of extracting oil from DWG using ethyl acetate solvent. A total of 9 bench-scale tests were conducted at three different temperatures using ethyl acetate as the solvent. DWG was intensely blended with solvent to perform the oil extraction. Experimentation and analysis was performed as set forth in Example 1. The results are shown in Tables 5, 6 and 7.

TABLE 5 summarizes the results of corn biomass oil extraction from DWG using ethyl acetate as solvent at a temperature of 35° C. At 35° C., the Specific Gravity of ethyl acetate may be 0.8848 and the content of ethyl acetate in water phase may be 45.15 g/L. The amount of DWG used in these tests may be normalized to 10 g for comparison purposes.

TABLE 5
Results of Oil Extraction from DWG using Ethyl Acetate as Solvent at 35° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| DWG1 | 3.3578 | 0.0734 | 1.0668 | 24.78 | 0.10 | 24.9 |
| DWG2* | 3.4081 | 0.0801 | 1.0511 | 24.66 | N/A | 25.2 |
| DWG3* | 3.1535 | 0.0759 | 0.9921 | 24.82 | N/A | 24.8 |
| Normalized | 10.0000 | 0.2186 | 3.0871 | 24.78 | 0.10 | 24.9 |
| to 10 g | | | | | | |

*not used in normalization

Normalized for 10.0000 g of DWG which gives water content of 6.9129 g and DM of 3.0871 g. The DM may be further divided into oil of 0.0031+0.2186=0.2217 g, protein of 0.7687+0.0119=0.7806 g and other DM of 2.0848 g. The combined extraction solution was allowed to settle into two phases. The solvent phase includes 118.93 ml or 105.2293 g ethyl acetate, 24.78 g/L×118.93 ml=2.9471 g water, 0.10 g/L.times.118.93 ml=0.0119 g protein and 0.2186 g oil, for a total of 108.4069 g, or 3.1776 g on an ethyl acetate free basis. The water-solid phase includes water equal to 6.9129−2.9471=3.9658 g, oil equal to 0.0031 g, protein equal to 24.9 wt %×3.0871=0.7687 g, other DM equal to 2.0848 g and ethyl acetate equal to 45.15 g/L×3.9658 ml=0.1791 g=0.2024 ml, for a total of 6.2328 g, or 2.2670 g on a water free basis.

TABLE 6 summarizes the results of corn-based biomass oil extraction from DWG using ethyl acetate as solvent at a higher temperature and in particular a temperature of 45° C. At 45° C., the Specific Gravity of ethyl acetate may be 0.8733 and the content of ethyl acetate in water phase may be 48.21 g/L. The amount of DWG used in these tests may be normalized to 10 g for comparison purposes.

TABLE 6
Results of Oil Extraction from DWG using Ethyl Acetate as Solvent at 45° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| DWG1 | 2.4359 | 0.0498 | 0.7739 | 26.20 | 0.10 | 25.10 |
| DWG2 | 2.2987 | 0.0490 | 0.7089 | 26.05 | N/A | 24.80 |
| DWG3 | 2.7657 | 0.0621 | 0.8701 | 26.09 | N/A | 25.00 |
| Average | 2.5001 | 0.0536 | 0.7843 | 26.11 | 0.10 | 24.97 |
| Normalized | 10.0000 | 0.2144 | 3.1371 | 26.11 | 0.10 | 24.97 |
| to 10 g | | | | | | |

Normalized for 10.0000 g of DWG which gives the water content of 6.8629 g and DM of 3.1371 g. The DM may be further divided into oil of 0.0031+0.2144=0.2175 g, protein of 0.7833+0.0160=0.7993 g and other DM of 2.1203 g. The combined extraction solution was allowed to settle into two phases. The solvent phase includes 159.84 ml or 139.5883 g ethyl acetate, 26.11 g/L×159.84 ml=4.1734 g water, 0.10 g/L×159.84 ml=0.0160 g protein and 0.2144 g oil, for a total of 143.9921 g, or 4.4038 g on an ethyl acetate free basis. The water-solid phase includes water equal to 6.8629−4.1734=2.6895 g, oil equal to 0.0031 g, protein equal to 24.97 wt %×3.1371=0.7833 g, other DM equal to 2.1203 g and ethyl acetate equal to 48.21 g/L×2.6895 ml=0.1297 g=0.1486 ml, for a total of 5.7259 g, or 3.0364 g on a water free basis.

TABLE 7 summarizes the results of corn-based biomass oil extraction from DWG using ethyl acetate as solvent at a higher temperature and in particular a temperature of 55° C. At 55° C., the Specific Gravity of ethyl acetate may be 0.8613 and the content of ethyl acetate in water phase may be 51.18 g/L. The amount of DWG used in these tests may be normalized to 10 g for comparison purposes.

TABLE 7
Results of Oil Extraction from DWG using Ethyl Acetate as Solvent at 55° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| DWG1 | 2.4801 | 0.0512 | 0.7879 | 27.03 | 0.12 | 24.6 |
| DWG2 | 2.4620 | 0.0542 | 0.7593 | 27.07 | N/A | 25.2 |
| DWG3 | 3.8099 | 0.0848 | 1.1986 | 27.12 | N/A | 24.9 |
| Average | 2.9173 | 0.0634 | 0.9153 | 27.07 | 0.12 | 24.9 |
| Normalized | 10.0000 | 0.2173 | 3.1375 | 27.07 | 0.12 | 24.9 |
| to 10 g | | | | | | |

Normalized for 10.0000 g of DWG which gives water content of 6.8625 g and DM of 3.1375 g. The DM may be further divided into oil of 0.0031+0.2173=0.2204 g, protein of 0.7812+0.0164=0.7976 g and other DM of 2.1195 g. The combined extraction solution was allowed to settle into two phases. The solvent phase includes 136.92 ml or 117.9292 g ethyl acetate, 27.07 g/L×136.92 ml=3.7064 g water, 0.12 g/L×136.92 ml=0.0164 g protein and 0.2173 g oil, for a total of 121.8693 g, or 3.9401 g on an ethyl acetate free basis. The water-solid phase includes water equal to 6.8625−3.7064=3.1561 g, oil equal to 0.0031 g, protein equal to 24.9 wt %×3.1375=0.7812 g, other DM equal to 2.1195 g and ethyl acetate equal to 51.18 g/L×3.1561 ml=0.1615 g=0.1875 ml, for a total of 6.2214 g, or 3.0653 g on a water free basis.

As indicated in the method, water phase samples were collected at three different temperatures: 35° C., 45° C. and 55° C. and analyzed using Gas Chromatography. Ethyl acetate content in the water phase was 45.15 g/L at 35° C., 48.21 g/L at 45° C. and 51.18 g/L at 55° C.

TABLE 8
Results of Oil Extraction Using Ethyl Acetate as Solvent

| | | | | Water in | Protein in | |
|---|---|---|---|---|---|---|
| | | | Oil Yield, | Solvent | Solvent | Protein in |
| | Wet | Oil | wt %, | Phase, | Phase, | Solids, wt %, |
| | Weight, g | Extracted, g | DM | g/L | g/L | DM |
| T = 35° C. | | | | | | |
| TS1 | 8.6963 | 0.1537 | 12.3513 | 24.59 | 0.12 | 27.5 |
| TS2 | 9.4857 | 0.1611 | 11.8682 | 25.02 | NA* | 27.8 |
| TS3 | 10.7799 | 0.1760 | 11.4093 | 24.73 | NA | 27.1 |
| DWG1 | 3.3578 | 0.0734 | 6.9706 | 24.78 | 0.10 | 24.9 |
| DWG2 | 3.4081 | 0.0801 | 7.4943 | 24.66 | NA | 25.2 |
| DWG3 | 3.1535 | 0.0759 | 7.6752 | 24.82 | NA | 24.8 |
| T = 45° C. | | | | | | |
| TS1 | 6.5279 | 0.1110 | 11.8831 | 26.21 | 0.12 | 27.7 |
| TS2 | 7.6276 | 0.1369 | 12.5424 | 26.14 | NA | 27.2 |
| TS3 | 9.3904 | 0.1516 | 11.2823 | 26.16 | NA | 27.3 |
| DWG1 | 2.4359 | 0.0498 | 6.5192 | 26.20 | 0.10 | 25.1 |
| DWG2 | 2.2987 | 0.049 | 6.7980 | 26.05 | NA | 24.8 |
| DWG3 | 2.7657 | 0.0621 | 7.1602 | 26.09 | NA | 25.0 |
| T = 55° C. | | | | | | |
| TS1 | 8.6939 | 0.1522 | 12.2337 | 27.08 | 0.13 | 27.5 |
| TS2 | 8.4482 | 0.1354 | 11.2003 | 26.89 | NA | 27.2 |
| TS3 | 9.4122 | 0.168 | 12.4731 | 27.11 | NA | 27.0 |
| DWG1 | 2.4801 | 0.0512 | 6.58352 | 27.03 | 0.12 | 24.6 |
| DWG2 | 2.4620 | 0.0542 | 7.01981 | 27.07 | NA | 25.2 |
| DWG3 | 3.8099 | 0.0848 | 7.09742 | 27.12 | NA | 24.9 |

*Not Analyzed

Ethyl acetate may be effective in extracting corn-based biomass oil out of TS and DWG, as the leftover oil content in the solids after extraction was too low to be quantified (<0.1 wt % DM). Furthermore, extraction results in oil and protein being separated into two different phases. The content of protein in the solvent phase was also very low, only about 0.1 g/L. The influence of temperature on oil extraction was not significant. There was no significant change in water content in the solvent phase either.

In this example, oil was extracted from corn-based biomass produced TS and DWG using ethyl acetate as one method of extraction. We expect ethyl acetate to achieve similar results with TS and DWG produced from other biomass materials.

### Example 5

### Isopropyl Acetate Extraction From TS

The following represents one method of extracting oil from TS using isopropyl acetate solvent. 18 bench-scale tests were conducted at six different temperatures using isopropyl acetate (IPA) as the solvent. TS was intensely blended with solvent to perform the oil extraction. Experimentation and analysis was performed as set forth in Example 1. For purposes of example, nine test results at three different temperatures are shown in Tables 9, 10 and 11.

TABLE 9 summarizes the results of corn-based biomass oil extraction from TS using isopropyl acetate as solvent at a temperature of 45° C. At 45° C., the Specific Gravity of isopropyl acetate may be 0.8475 and the content of isopropyl acetate in water phase may be 27.86 g/L. The amount of TS used in these tests may be normalized to 10 g for comparison purposes.

TABLE 9
Results of Oil Extraction from TS using Isopropyl Acetate as Solvent at 45° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| TS1 | 4.9386 | 0.0886 | 0.7102 | 23.25 | 0.13 | 26.9 |
| TS2 | 5.4607 | 0.0919 | 0.7771 | 23.11 | N/A | 27.3 |
| TS3 | 5.5273 | 0.0962 | 0.7915 | 23.37 | N/A | 27.6 |
| Average | 5.3089 | 0.0922 | 0.7596 | 23.24 | 0.13 | 27.3 |
| Normalized | 10.0000 | 0.1737 | 1.4308 | 23.24 | 0.13 | 27.3 |
| to 10 g | | | | | | |

Normalized for 10.0000 g of TS which gives water content of 8.5692 g and DM content of 1.4308 g. The DM may be further divided into oil of 0.0014+0.1737=0.1751 g, protein of 0.3906+0.0098=0.4004 g and other DM of 0.8553 g. The combined extraction solution was allowed to settle into two phases. The solvent phase includes 75.12 ml or 63.6642 g isopropyl acetate, 23.24 g/L×75.12 ml=1.7458 g water, 0.13 g/L×75.12 ml=0.0098 g protein and 0.1737 g oil, for a total of 65.5935 g, or 1.9293 g on an isopropyl acetate free basis. The water-solid phase includes water equal to 8.5692−1.7458=6.8234 g, oil equal to 0.0014 g, protein equal to 27.3 wt %×1.4308=0.3906 g, other DM equal to 0.8553 g and isopropyl acetate equal to 27.86 g/L×6.8234 ml=0.901 g=0.2243 ml, for a total of 8.2608 g, or 1.4374 g on a water free basis.

TABLE 10 summarizes the results of corn-based biomass oil extraction from TS using isopropyl acetate as solvent at a higher temperature, namely a temperature of 65° C. At 65° C., the Specific Gravity of isopropyl acetate may be 0.8240 and the content of isopropyl acetate in water phase may be 30.82 g/L. The amount of TS used in these tests may be normalized to 10 g for comparison purposes.

TABLE 10
Results of Oil Extraction from TS using Isopropyl Acetate as Solvent at 65° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| TS1 | 6.6895 | 0.1148 | 0.9620 | 26.02 | 0.13 | 27.1 |
| TS2 | 6.4477 | 0.1114 | 0.9175 | 25.79 | N/A | 26.9 |
| TS3 | 5.4108 | 0.0898 | 0.7748 | 26.13 | N/A | 27.3 |
| Average | 6.1827 | 0.1053 | 0.8848 | 25.98 | 0.13 | 27.1 |
| Normalized | 10.0000 | 0.1703 | 1.4311 | 25.98 | 0.13 | 27.1 |
| to 10 g | | | | | | |

Normalized for 10.0000 g of TS which gives water content of 8.5689 g and DM of 1.4311 g. The DM may be further divided into oil of 0.0014+0.1703=0.1717 g, protein of 0.3878+0.0084=0.3962 g and other DM of 0.8632 g. The combined extraction solution was allowed to settle into two phases. The solvent phase includes 64.44 ml or 53.0986 g isopropyl acetate, 25.98 g/L×64.44 ml=1.6742 g water, 0.13 g/L×64.44 ml=0.0084 g protein and 0.1703 g oil, for a total of 54.9515 g, or 1.8529 g on an isopropyl acetate free basis. The water-solid phase includes water equal to 8.5689−1.6742=6.8947 g, oil equal to 0.0014 g, protein equal to 271 wt %×1.4311=0.3878 g, other DM equal to 0.8632 g and isopropyl acetate equal to 30.82 g/L×6.8947 ml=0.2125 g=0.2578 ml, for a total of 8.3596 g, or 1.4649 g on a water free basis.

TABLE 11 summarizes the results of corn-based biomass oil extraction from TS using isopropyl acetate as solvent at a higher temperature, namely a temperature of 80° C. At 80° C., the Specific Gravity of isopropyl acetate may be 0.8058 and the content of isopropyl acetate in water phase may be 33.21 g/L. The amount of TS used in these tests may be normalized to 10 g for comparison purposes.

TABLE 11
Results of Oil Extraction from TS using Isopropyl Acetate as Solvent at 80° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| TS1 | 7.0079 | 0.1133 | 1.0077 | 29.13 | 0.15 | 27.0 |
| TS2 | 6.4712 | 0.1113 | 0.9209 | 29.42 | N/A | 26.8 |
| TS3 | 6.8291 | 0.1211 | 0.9779 | 29.19 | N/A | 27.4 |
| Average | 6.7698 | 0.1152 | 0.9688 | 29.25 | 0.15 | 27.1 |
| Normalized | 10.0000 | 0.1702 | 1.4311 | 29.25 | 0.15 | 27.1 |
| to 10 g | | | | | | |

Normalized for 10.0000 g of TS which gives water content of 8.5689 g and DM of 1.4311 g. The DM may be further divided into oil of 0.0014+0.1702=0.1716 g, protein of 0.3878+0.0088=0.3966 g and other DM of 0.8629 g. The combined extraction solution was allowed to settle into two phases. The solvent phase includes 58.80 ml or 47.3810 g isopropyl acetate, 29.25 g/L×58.80 ml=1.7199 g water, 0.15 g/L×58.80 ml=0.0088 g protein and 0.1702 g oil, for a total of 49.2799 g, or 1.8989 g on an isopropyl acetate free basis. The water-solid phase includes water equal to 8.5689−1.7199=6.8490 g, oil equal to 0.0014 g, protein equal to 27.1 wt %×1.4311=0.3878 g, other DM equal to 0.8629 g and isopropyl acetate equal to 33.21 g/L×6.8490 ml=0.2275 g=0.2823 ml, for a total of 8.3286 g, or 1.4796 g on a water free basis.

From the foregoing, it may be understood that, similar to ethyl acetate, isopropyl acetate may be also effective in extracting corn-based biomass oil from TS. Moreover, temperature has little or no effect on the effectiveness of isopropyl acetate solvent in removal of oil. The amount of isopropyl acetate in water phase may be smaller in the above results than ethyl acetate due to the decreased solubility of isopropyl acetate in water.

In this example, isopropyl acetate was used to extract corn-based biomass oil from TS as one method of extraction. We expect isopropyl acetate to achieve similar results in extracting oil from TS derived from other biomass materials.

### Example 6

### Oil Extraction From DWG Using Isopropyl Acetate

The following represents one method of extracting oil from DWG using isopropyl acetate solvent. 18 bench-scale tests were conducted at six different temperatures using isopropyl acetate as the solvent. DWG was intensely blended with solvent to perform the oil extraction. Experimentation and analysis was performed as set forth in Example 1. As examples, nine test results at three different temperatures are shown in Tables 12, 13 and 14.

TABLE 12 summarizes the results of corn-based biomass oil extraction from DWG using isopropyl acetate as solvent at a temperature of 45° C. At 45° C., the Specific Gravity of isopropyl acetate may be 0.8475 and the content of isopropyl acetate in water phase may be 27.86 g/L. The amount of DWG used in these tests may be normalized to 10 g for comparison purposes.

TABLE 12
Results of Oil Extraction from DWG using Isopropyl Acetate as Solvent at 45° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| DWG1 | 2.3059 | 0.0461 | 0.7326 | 23.78 | 0.10 | 25.10 |
| DWG2 | 2.4705 | 0.0545 | 0.7619 | 23.61 | N/A | 24.60 |
| DWG3 | 2.1957 | 0.0492 | 0.6908 | 23.34 | N/A | 24.90 |
| Average | 2.3240 | 0.0499 | 0.7284 | 23.58 | 0.10 | 24.87 |
| Normalized | 10.0000 | 0.2147 | 3.1343 | 23.58 | 0.10 | 24.87 |
| to 10 g | | | | | | |

Normalized for 10.0000 g of DWG which gives water content of 6.8657 g and DM of 3.1343 g. The DM may be further divided into oil of 0.0031+0.2147=0.2178 g, protein of 0.7795+0.0172=0.7967 g and other DM of 2.1198 g. The combined extraction solution was allowed to settle into two phases. The solvent phase includes 172.03 ml or 145.7954 g isopropyl acetate, 23.58 g/L×172.03 ml=4.0565 g water, 0.10 g/L×172.03 ml=0.0172 g protein and 0.2147 g oil, for a total of 150.0838 g, or 4.2884 g on an isopropyl acetate free basis. The water-solid phase includes water equal to 6.8657−4.0565=2.8092 g, oil equal to 0.0031 g, protein equal to 24.87 wt %×3.1343=0.7795 g, other DM equal to 2.1198 g and isopropyl acetate equal to 27.86 g/L×2.8092 ml=0.0783 g=0.0924 ml, for a total of 5.7899 g, or 2.9807 g on a water free basis.

TABLE 13 summarizes the results of corn-based biomass oil extraction from DWG using isopropyl acetate as solvent at a higher temperature, namely a temperature of 65° C. At 65° C., the Specific Gravity of isopropyl acetate may be 0.8240 and the content of isopropyl acetate in water phase may be 31.56 g/L. The amount of DWG used in these tests may be normalized to 10 g for comparison purposes.

TABLE 13
Results of Oil Extraction from DWG using Isopropyl Acetate as Solvent at 65° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| DWG1 | 2.4786 | 0.0512 | 0.7875 | 26.06 | 0.12 | 24.7 |
| DWG2 | 2.4558 | 0.0529 | 0.7574 | 25.91 | N/A | 25.1 |
| DWG3 | 3.8104 | 0.0841 | 1.1988 | 26.12 | N/A | 24.8 |
| Average | 2.9149 | 0.0627 | 0.9146 | 26.03 | 0.12 | 24.9 |
| Normalized | 10.0000 | 0.2151 | 3.1375 | 26.03 | 0.12 | 24.9 |
| to 10 g | | | | | | |

Normalized for 10.0000 g of DWG which gives water content of 6.8625 g and DM of 3.1375 g. The DM may be further divided into oil of 0.0031+0.2151=0.2182 g, protein of 0.7812+0.0165=0.7977 g and other DM of 2.1216 g. The combined extraction solution was allowed to settle into two phases. The solvent phase includes 137.10 ml or 112.9703 g isopropyl acetate, 26.03 g/L×137.10 ml=3.5687 g water, 0.12 g/L.times.137.10 ml=0.0165 g protein and 0.2151 g oil, for a total of 116.7706 g, or 3.8003 g on an isopropyl acetate free basis. The water-solid phase includes water equal to 6.8625−3.5687=3.2938 g, oil equal to 0.0031 g, protein equal to 24.9 wt %×3.1375=0.7812 g, other DM equal to 2.1216 g and isopropyl acetate equal to 31.56 g/L×3.2938 ml=0.1039 g=0.1261 ml, for a total of 6.3036 g, or 3.0098 g on a water free basis.

TABLE 14 summarizes the results of corn-based biomass oil extraction from DWG using isopropyl acetate as solvent at a higher temperature, namely a temperature of 80° C. At 80° C., the Specific Gravity of isopropyl acetate may be 0.8058 and the content of isopropyl acetate in water phase may be 33.21 g/L. The amount of DWG used in these tests may be normalized to 10 g for comparison purposes.

TABLE 14
Results of Oil Extraction from DWG using Isopropyl Acetate as Solvent at 80° C.

| | | | | Water in | Protein in | Protein in |
|---|---|---|---|---|---|---|
| | | | | Solvent | Solvent | Solids, |
| | Wet | Oil | | Phase, | Phase, | wt %, |
| | Weight, g | Extracted, g | DM, g | g/L | g/L | DM |
| DWG1 | 2.3313 | 0.0499 | 0.7407 | 29.21 | 0.13 | 24.9 |
| DWG2 | 2.0498 | 0.0474 | 0.6322 | 28.96 | N/A | 24.8 |
| DWG3 | 2.7838 | 0.0615 | 0.8758 | 29.07 | N/A | 25.3 |
| Average | 2.3883 | 0.0529 | 0.7495 | 29.08 | 0.13 | 25.0 |
| Normalized | 10.0000 | 0.2215 | 3.1382 | 29.08 | 0.13 | 25.0 |
| to 10 g | | | | | | |

Normalized for 10.0000 g of DWG which gives the water content of 6.8618 g and DM of 3.1382 g. The DM may be further divided into oil of 0.0031+0.2215=0.2246 g, protein of 0.7846+0.0218=0.8064 g and other DM of 2.1072 g. The combined extraction solution was allowed to settle into two phases. The solvent phase includes 167.40 ml or 134.8909 g isopropyl acetate, 29.08 g/L×167.40 ml=4.8680 g water, 0.13 g/L×167.40 ml=0.0218 g protein and 0.2215 g oil, for a total of 140.0022 g, or 5.1113 g on an isopropyl acetate free basis. The water-solid phase includes water equal to 6.8618−4.8680=1.9938 g, oil equal to 0.0031 g, protein equal 25.0 wt %×3.1382=0.7846 g, other DM equal to 2.1072 g and isopropyl acetate equal to 33.21 g/L×1.9938 ml=0.0662 g=0.0822 ml, for a total of 4.9549 g, or 2.9611 g on a water free basis. As indicated in the method, water phase samples were collected at six different temperatures: 45° C., 55° C., 65° C., 70° C., 80° C. and 90° C. The Gas Chromatography results of these samples showed that isopropyl acetate content in the water phase was 27.86 g/L at 45° C., 29.31 g/L at 55° C., 30.82 g/L at 65° C., 31.56 g/L at 70° C., 33.21 g/L at 80° C. and 50.35 g/L at 90° C.

TABLE 15
Results of Oil Extraction Using Isopropyl Acetate as Solvent

| | | | | Water in | Protein in | Protein in |
|---|---|---|---|---|---|---|
| | Wet | Oil | Oil Yield, | Solvent | Solvent | Solids, |
| | Weight, g | Extracted, g | wt %, DM | Phase, g/L | Phase, g/L | wt %, DM |
| T = 45° C. | | | | | | |
| TS1 | 4.9386 | 0.0886 | 12.5371 | 23.25 | 0.13 | 26.9 |
| TS2 | 5.4607 | 0.0919 | 11.7609 | 23.11 | NA* | 27.3 |
| TS3 | 5.5273 | 0.0962 | 12.1634 | 23.37 | NA | 27.6 |
| DWG1 | 2.3059 | 0.0461 | 6.3753 | 23.78 | 0.10 | 25.1 |
| DWG2 | 2.4705 | 0.0545 | 7.0349 | 23.61 | NA | 24.6 |
| DWG3 | 2.1957 | 0.0492 | 7.1460 | 23.34 | NA | 24.9 |
| T = 55° C. | | | | | | |
| TS1 | 8.2875 | 0.1364 | 11.5018 | 24.29 | 0.13 | 27.4 |
| TS2 | 6.1704 | 0.1072 | 12.1418 | 23.81 | NA | 26.8 |
| TS3 | 5.1213 | 0.0849 | 11.5841 | 24.16 | NA | 27.1 |
| DWG1 | 3.0825 | 0.0645 | 6.6721 | 24.08 | 0.11 | 25.0 |
| DWG2 | 3.1683 | 0.0688 | 6.9243 | 24.15 | NA | 24.5 |
| DWG3 | 2.3815 | 0.0514 | 6.8827 | 23.98 | NA | 25.2 |
| T = 65° C. | | | | | | |
| TS1 | 6.6895 | 0.1148 | 11.9920 | 26.02 | 0.13 | 27.1 |
| TS2 | 6.4477 | 0.1114 | 12.0732 | 25.79 | NA | 26.9 |
| TS3 | 5.4108 | 0.0898 | 11.5975 | 26.13 | NA | 27.3 |
| DWG1 | 2.4786 | 0.0512 | 6.5869 | 26.06 | 0.12 | 24.7 |
| DWG2 | 2.4558 | 0.0529 | 6.8692 | 25.91 | NA | 25.1 |
| DWG3 | 3.8104 | 0.0841 | 7.0383 | 26.12 | NA | 24.8 |
| T = 70° C. | | | | | | |
| TS1 | 5.978 | 0.1019 | 11.9118 | 27.91 | 0.13 | 27.6 |
| TS2 | 5.1124 | 0.0837 | 11.4409 | 27.24 | NA | 27.7 |
| TS3 | 6.3153 | 0.1116 | 12.3489 | 27.37 | NA | 27.2 |
| DWG1 | 2.0013 | 0.0393 | 6.2618 | 27.88 | 0.12 | 24.8 |
| DWG2 | 2.5419 | 0.0525 | 6.5860 | 27.65 | NA | 25.1 |
| DWG3 | 2.6141 | 0.0559 | 6.8188 | 27.81 | NA | 24.9 |
| T = 80° C. | | | | | | |
| TS1 | 7.0079 | 0.1133 | 11.2980 | 29.13 | 0.15 | 27.0 |
| TS2 | 6.4712 | 0.1113 | 12.0191 | 29.42 | NA | 26.8 |
| TS3 | 6.8291 | 0.1211 | 12.3919 | 29.19 | NA | 27.4 |
| DWG1 | 2.3313 | 0.0499 | 6.8253 | 29.21 | 0.13 | 24.9 |
| DWG2 | 2.0498 | 0.0474 | 7.3737 | 28.96 | NA | 24.8 |
| DWG3 | 2.7838 | 0.0615 | 7.0446 | 29.07 | NA | 25.3 |
| T = 90° C. | | | | | | |
| TS1 | 5.2885 | 0.0907 | 11.9849 | 52.01 | 0.15 | 26.9 |
| TS2 | 5.6949 | 0.1005 | 12.3322 | 51.85 | NA | 27.0 |
| TS3 | 7.7857 | 0.1262 | 11.3271 | 52.32 | NA | 27.3 |
| DWG1 | 2.0734 | 0.0419 | 6.4439 | 52.04 | 0.13 | 24.6 |
| DWG2 | 3.1944 | 0.0639 | 6.3787 | 52.07 | NA | 24.9 |
| DWG3 | 2.2790 | 0.0488 | 6.8280 | 52.16 | NA | 25.1 |

*Not Analyzed

From the foregoing, it may be understood that, similar to ethyl acetate, isopropyl acetate may be effective in extracting corn-based biomass oil from DWG as well as TS, as the leftover oil content in the solids after extraction was too low to be quantified (<0.1 wt % DM). The content of protein in the solvent phase was also very low, only about 0.1 g/L. In addition, temperature has little or no effect on the effectiveness of the isopropyl acetate solvent in removal of oil. The amount of isopropyl acetate in water phase may be smaller in the above results than ethyl acetate due to the decreased solubility of isopropyl acetate in water.

In this example, isopropyl acetate was used to extract corn-based biomass oil from DWG as one method of extraction. We expect isopropyl acetate to achieve similar results in extracting oil from DWG derived from other biomass materials.

### Example 7

### Oil Extraction From DDGS Using Ethyl Acetate

The following represents one method of extracting oil from DDGS using ethyl acetate solvent at 25° C. Bench-scale tests were conducted at three different solvent to DDGS ratios. DDGS was intensely blended with solvent to perform the oil extraction. Specifically, 8.6 g of DDGS was intensely blended respectively, with 10 ml, 20 ml and 30 ml solvent to arrive at the different ratios. Three extractions were performed for each ratio. Experimentation and analysis were performed similar as set forth in Example 1. The results are listed in TABLE 16.

TABLE 16 summarizes the results of corn-based biomass oil extraction from DDGS using ethyl acetate as solvent at a temperature of 25° C. At 25° C., the Specific Gravity of ethyl acetate may be 0.8963, the content of ethyl acetate in water phase may be 42.09 g/L and water content in the solvent phase may be 23.39 g/L. 8.6 g of DDGS was normalized to 10 g in these tests for comparison purposes.

TABLE 16
Results of Oil Extraction from DDGS using
Ethyl Acetate as Solvent at 25° C.

| DDGS 8.6 g | 1:1 | 2:1 | 3:1 |
|---|---|---|---|
| Oil (g) | 10 ml | 20 ml | 30 ml |
| | Solvent each | Solvent each | Solvent each |
| 1st extraction | 0.0723 | 0.2425 | 0.3440 |
| 2nd extraction | 0.3086 | 0.2944 | 0.2809 |
| 3rd extraction | 0.1934 | 0.1375 | 0.1003 |
| Total | 0.5743 | 0.6744 | 0.7252 |

Normalized to 10 g of Corn-based Biomass:

| DDGS 10 g | 1:1 | 2:1 | 3:1 |
|---|---|---|---|
| Oil (g) | 11.63 ml | 23.26 ml | 34.88 ml |
| | Solvent each | Solvent each | Solvent each |
| 1st extraction | 0.0841 | 0.2820 | 0.4000 |
| 2nd extraction | 0.3588 | 0.3423 | 0.3266 |
| 3rd extraction | 0.2249 | 0.1599 | 0.1166 |
| Total | 0.6678 | 0.7842 | 0.8432 |

Normalized for 10.0000 g of DDGS which gives water content of 1.0000 g and DM of 9.0000 g. The DM may be further divided into oil of 0.9281 g, protein of 3.1270 g and other of 4.9449 g DM. The solvent used for extraction may be 104.65 ml ethyl acetate. However, the maximum amount of water content in 104.65 ml ethyl acetate may be 23.89 g/L×104.65 ml=2.4478 g at 25° C. which may be much higher than 1.0000 g water. Therefore, there may be no water phase present. This process results in 0.8432 g oil extracted and 0.0849 g oil remaining after the 3^{rd} extraction with 34.88 ml of solvent used for each extraction.

The oil content in the DDGS was measured to be 0.9281 g. Therefore, 90.85 wt % of the oil was extracted after the 3^{rd} extraction with 3:1 solvent to DDGS ratio. For higher oil recovery efficiency, both a 4^{th} extraction and higher solvent to DDGS ratio may be utilized due to the high oil content in DDGS.

In this example, ethyl acetate was used to extract corn-based biomass oil from DDGS as one method of extraction. We expect ethyl acetate to achieve similar results in extracting oil from DDGS derived from other biomass materials.

### Example 8

### Oil Extraction From Milled Corn-Based Biomass Using Ethyl Acetate as Solvent

The following represents one method of extracting oil from milled corn-based biomass using ethyl acetate solvent. Bench-scale tests were conducted at three different solvent to milled corn-based biomass ratios and are listed in Table 17. 8.6 g of milled corn-based biomass was intensely blended respectively, with 10 ml, 20 ml and 30 ml solvent to arrive at the different ratios. Three extractions were performed for each ratio. Experimentation and analysis were performed similar as set forth in Example 1.

TABLE 17 illustrates the results of corn-based biomass oil extraction by use of ethyl acetate solvent at a temperature of 25° C. At 25° C., the Specific Gravity of ethyl acetate may be 0.8963, the content of ethyl acetate in water phase may be 42.09 g/L and the water content in solvent phase may be 23.39 g/L.

TABLE 17
Results of Oil Extraction from Milled Corn Biomass using
Ethyl Acetate as Solvent at 25° C.

| Corn 8.6 g | 1:1 | 2:1 | 3:1 |
|---|---|---|---|
| Oil (g) | 10 ml | 20 ml | 30 ml |
| | Solvent each | Solvent each | Solvent each |
| 1st extraction | 0.1035 | 0.1612 | 0.2040 |
| 2nd extraction | 0.0974 | 0.0921 | 0.0917 |
| 3rd extraction | 0.0467 | 0.0315 | 0.0269 |
| Total | 0.2486 | 0.2848 | 0.3226 |

The 8.6 g of milled corn-based biomass may be normalized to
10 g in order to compare other corn-based biomass oil extraction
results from other tests. Normalized to 10 g of corn-based biomass:

| Corn 10 g | 1:1 | 2:1 | 3:1 |
|---|---|---|---|
| Oil (g) | 11.63 ml | 23.26 ml | 34.88 ml |
| | Solvent each | Solvent each | Solvent each |
| 1st extraction | 0.1203 | 0.1874 | 0.2372 |
| 2nd extraction | 0.1133 | 0.1071 | 0.1066 |
| 3rd extraction | 0.0543 | 0.0366 | 0.0313 |
| Total | 0.2879 | 0.3311 | 0.3751 |

For 10.0000 g of milled corn-based biomass, this gives water content of 1.5000 g and DM of 8.5000 g. The DM can be further divided into 0.3962 g oil, 0.8583 g protein and 7.2455 g other DM. The solvent used for extraction may be 104.65 ml ethyl acetate. However, the maximum amount of water content in 104.65 ml ethyl acetate may be 23.39 g/L×104.65 ml=2.4478 g at 25° C. which may be much higher than 1.5000 g. Therefore, there may be no water phase present. Oil extracted from the 3:1 mixture was 0.3751 g, with oil remaining of 0.0211 g.

The corn-based biomass oil content in 10 g of milled corn-based biomass may be measured as 0.3962 g and the oil extracted from the 3:1 solvent to milled corn-based biomass ratio after the 3^{rd} extraction may be 0.3751 g. Therefore, 94.67 wt % of the oil was recovered after the 3^{rd} extraction with 3:1 solvent to milled corn-based biomass ratio. For higher oil recovery efficiency, either a 4^{th} extraction or higher solvent to milled corn-based biomass ratio may be required.

In this example, ethyl acetate was used as solvent to extract corn-based biomass oil from milled corn-based biomass as one method of extraction. We expect ethyl acetate solvent to achieve similar results in extracting oil from other types of milled biomass materials.

### Example 9

### Oil Extraction From DWG Using Ethyl Acetate Solvent

When the DWG stream (35 wt % solids, balance primarily water) may be blended with a solvent, ethyl acetate in this case, two phases form: (1) The upper phase comprises of corn-based biomass oil in ethyl acetate which still contains a small amount of water; (2) The lower phase comprises primarily of water and solids (35 wt %) with a small amount of ethyl acetate—typically around 7 wt % at the temperatures discussed herein.

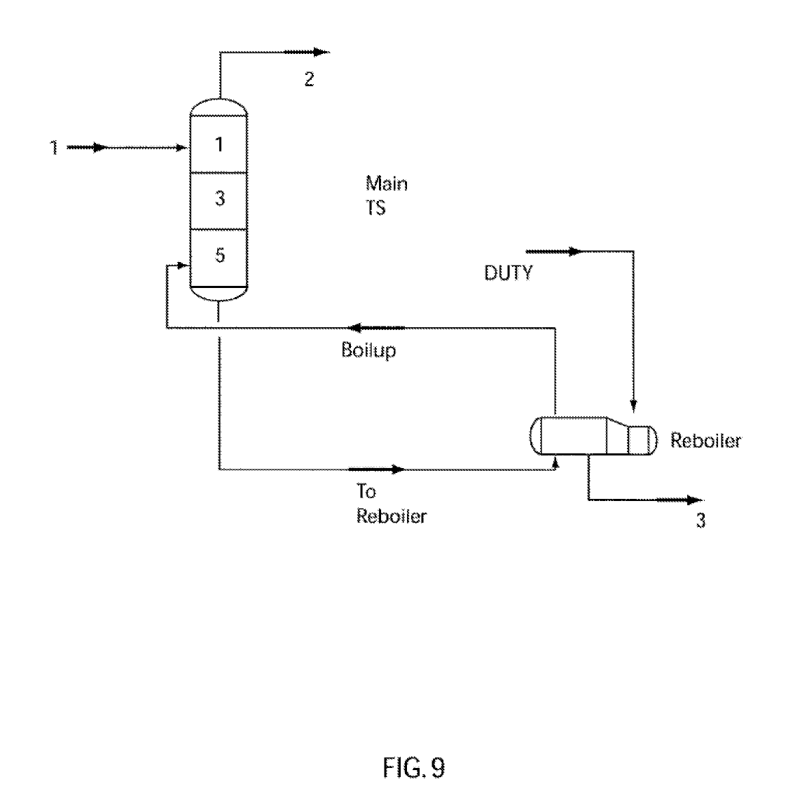

This lower phase may be decanted, preheated to about 77° C. and then directed into a reboiler desorption unit. As shown in FIG. 9, a detailed process model may be used to simulate the operation of the reboiler desorption unit for bench scale analysis. Five stages were used in the unit so that the ethyl acetate content in the third stream stays less than 1 ppm by weight. Ethyl acetate content of less than 1 ppm has significant advantages, as the ethyl acetate losses are kept to a minimum and no wastewater treatment may be required.

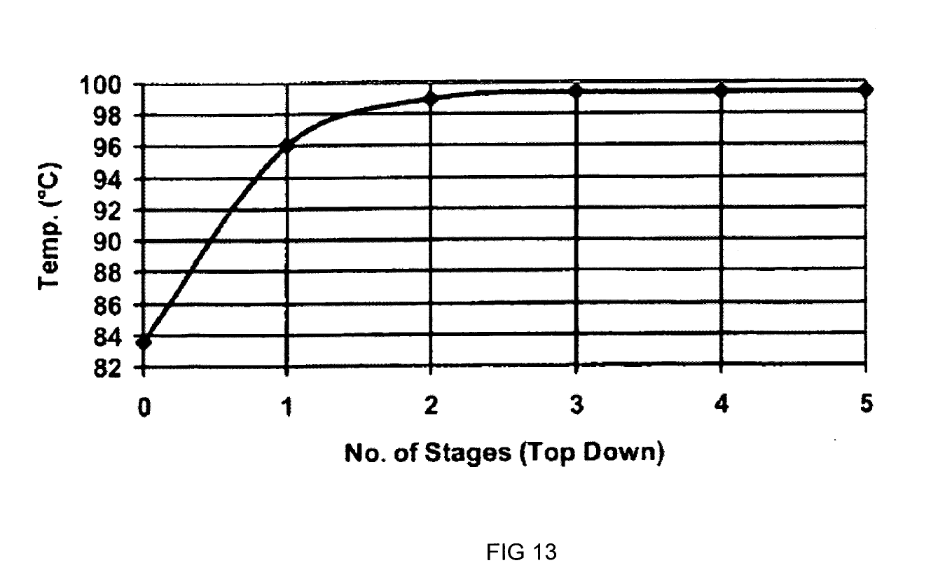

FIG. 13 and the tables below show the simulated results of material streams, temperature profiles, net liquid rates and net vapor rates in the five-stage reboiler desorption unit.

TABLE 18
5-STAGE REBOILER DESORPTION UNIT PROFILES

| | Name of Stream | | | | |
|---|---|---|---|---|---|
| | 2 | To Reboiler | Boil up | 3 | 1 |
| Vapor Fraction | 1.000 | 0.00 | 1.00 | 0.00 | 0.00 |
| Temperature (° C.) | 83.590 | 99.41 | 99.41 | 99.41 | 77.00 |
| Pressure (psia) | 14.500 | 14.50 | 14.50 | 14.50 | 14.50 |
| Molar Flow (lbmole/hr) | 7.726 | 227.30 | 14.60 | 212.70 | 220.50 |
| Mass Flow (lb/hr) | 381.200 | 4,095.00 | 263.00 | 3,832.00 | 4,214.00 |
| Liquid Volume Flow | 28.300 | 281.00 | 18.04 | 262.90 | 291.20 |
| (barrel/day) | | | | | |
| Heat Flow (kJ/h) | −1.087e+006 | −2.730e+007 | −1.497e+006 | −2.555e+007 | =2.689e+007 |

| | | Pressure | | Net Liquid | Net Vapor |
|---|---|---|---|---|---|
| | Stage | (psia) | Temp (° C.) | (lb/mole/hr) | (lb/mole/hr) |
| 1_Main TS | 0 | 14.50 | 83.59 | 221.9 | 7.726 |
| 2_Main TS | 1 | 14.50 | 96.09 | 226.1 | 9.159 |
| 3_Main TS | 2 | 14.50 | 98.97 | 227.2 | 13.36 |
| 4_Main TS | 3 | 14.50 | 99.36 | 227.3 | 14.42 |
| 5_Main TS | 4 | 14.50 | 99.41 | 227.3 | 14.57 |
| Reboiler | 5 | 14.50 | 99.41 | 212.7 | 14.60 |

It should be noted that only a small boil up stream which accounts for 6.4 mole % (6.5 wt %) of the reboiler feed stream may be needed to desorb out all the ethyl acetate in the reboiler desorption unit feed stream. As a result, the process requires a very small energy demand for the unit.

Stream 2 may be preferably about 3.5 mole % (14.5 wt %) of the feed stream to the unit and preferably contains 79.8 wt % ethyl acetate and 20.2 wt % water. Stream 2 may be recycled back into the mixer unit operation where the corn-based biomass oil may be extracted from DWG by subjecting the DWG to an ethyl acetate solvent to produce an extraction solution containing corn-based biomass oil.

The same process consideration also applies for deoiling the TS that may be available in the beer still bottoms.

### Example 10

### Generation of Power and Steam by Combustion of Deoiled DWG

Deoiled DWG has several advantages, one of which may be the generation of power and steam by combustion.

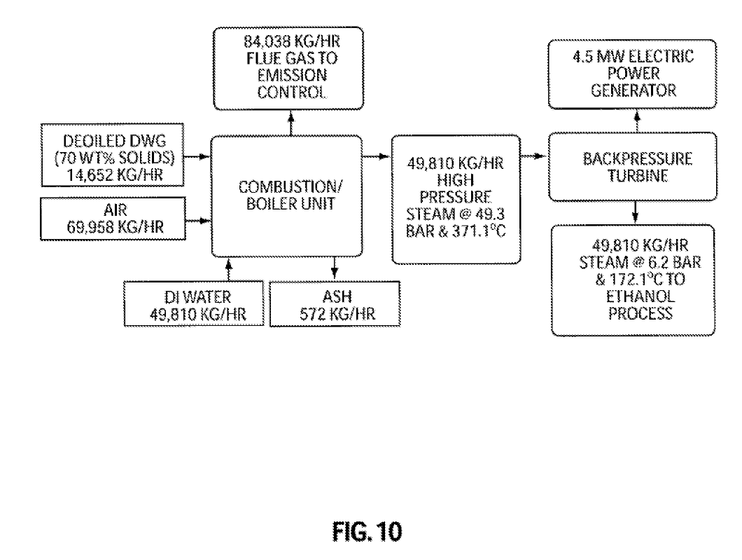

A stream of 14,652 kg/hr (32,303 lb/hr) of deoiled DWG with a moisture level of 30 wt % or 70 wt % solids may be fed into a combustion/boiler unit such as a type offered by KMW Systems, Inc. (identified above) including but not limited to an English boiler available from KMW Systems. The unit may be air blown and works at atmospheric pressure. A deionized water stream of 49,810 kg/hr and an air stream of 69,958 kg/hr are also fed into the combustion/boiler unit. The flue gas may be directed through a compact high efficiency boiler where high pressure steam at 49.3 bar (715 psia) and 371.1° C. (700° F.) may be produced. 84,038 kg/hr flue gas also exits the combustion/boiler unit toward an emission control unit. Ash may be also produced at approximately 572 kg/hr. This steam may be then fed into a backpressure turbine, which may be coupled to an electric power generator. Typical steam turbines may be available for this application from Dresser-Rand Murray (Houston, Tex.) and electric generators may be available from international companies like General Electric (Fairfield, Conn.). As a result of the foregoing, 4.5 MWH of electric power, commensurate with the pressure drop across the backpressure turbine, may be produced. 49,810 kg/hr (109,812 lb/hr) of exhaust steam at 6.2 bar (90 psia) and 172.1° C. (342° F.) may be also available and may be used for example, for the low pressure steam requirements of and may be thus routed to the ethanol production process. The pressure can be specifically calibrated to maximize the use of this steam for the beer stills. A block diagram schematic of this application may be shown in FIG. 10. In one aspect, in order to produce higher electrical power, the steam pressure and temperature can be increased to 63.1 bar (915 psia) and 386.0° C. (727° F.) so as to produce 5.0 MWH. More preferably, the combustion/boiler unit, the steam pressure and temperature can be increased to 87.2 bar (1,265 psia) and 411.4° C. (773° F.), to increase output, including but not limited to an electrical power output increase from 5.0 to 5.6 MW.

### Example 11

### Production of Syngas

As indicated herein, deoiled DWG may also be gasified to produce syngas rich in hydrogen and other products therefrom, such as anhydrous ammonia, DME, other alcohols and biofuels, or the like.

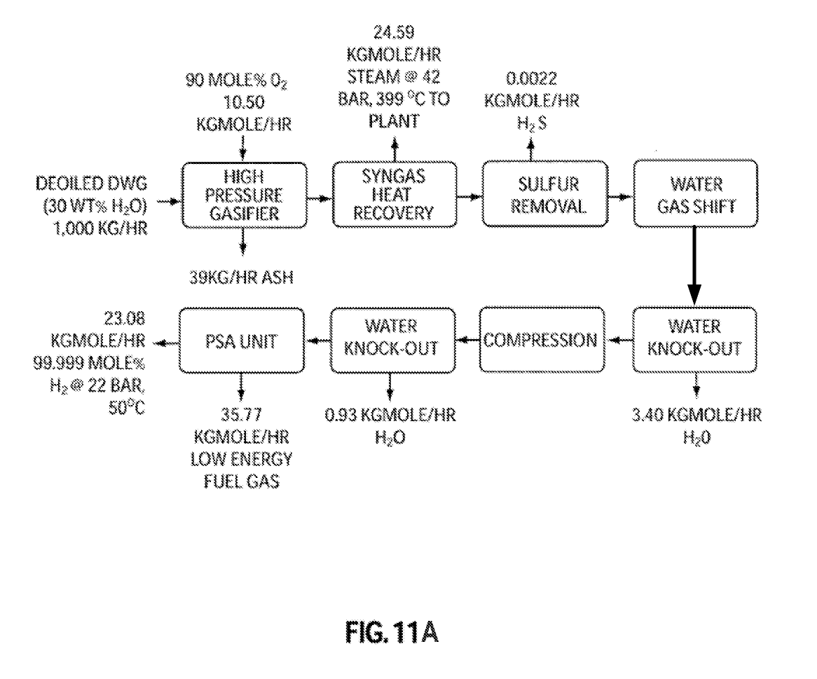

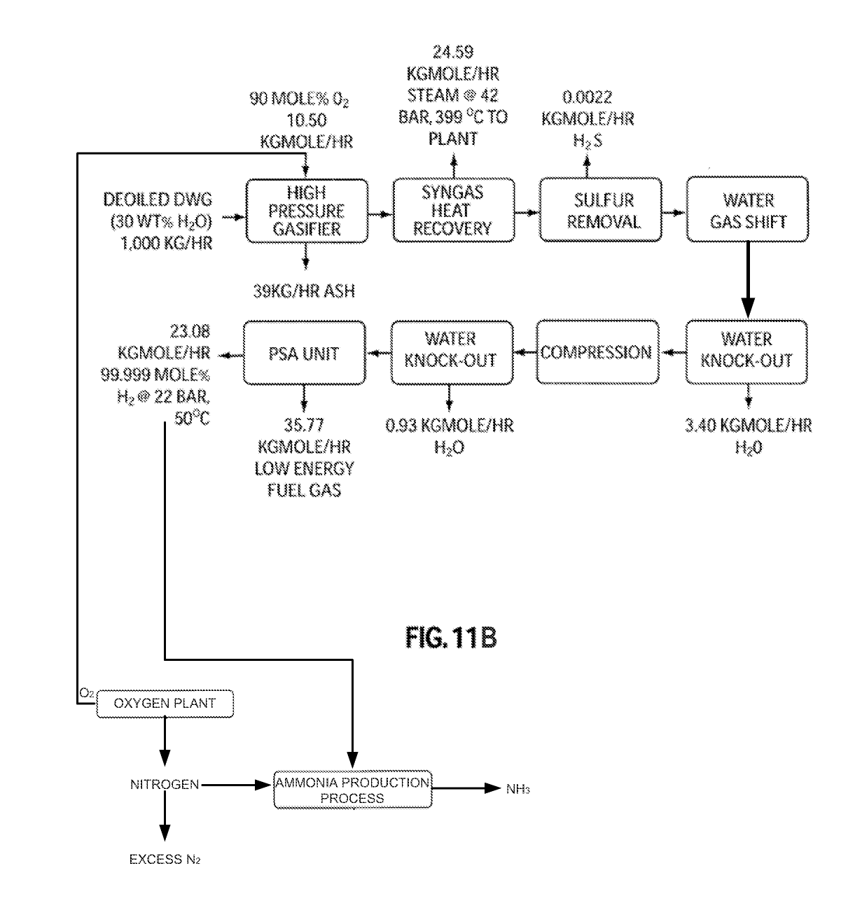

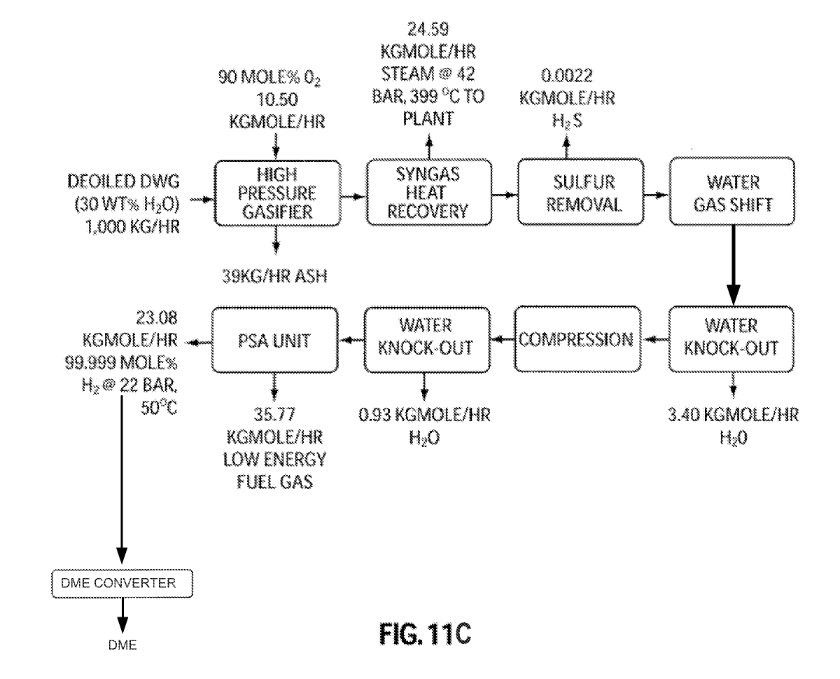

A block diagram schematic of the gasification process may be shown in FIG. 11A. 1,000 kg/hr of deoiled DWG with a moisture level of 30 wt % may be fed along with an oxidant containing 10.50 kgmole/hr of 90 mole % oxygen and 10 mole % nitrogen into a gasifier. The gasifier may be maintained at 4 bar and 962.67.degree. C. 63.18 kgmole/hr of syngas may be produced. 39 kg/hr of ash may be also produced. Syngas composition according to the foregoing may be detailed in TABLE 19.

TABLE 19
SYNGAS COMPOSITION

| Composition | Mole % |
|---|---|
| CH₄ | 0.01 |
| CO₂ | 17.14 |
| N₂ | 1.91 |
| CO | 23.67 |
| H₂ | 28.54 |
| H₂S (ppm) | 35 |
| H₂O | 28.72 |
| TOTAL | 100.00 |
| Pressure, Bar | 4.00 |
| Temperature, ° C. | 926.67 |
| HHV, BTU/SCF | 169 (wet), 237 (dry) |
| Kcal/M³ | 1,504 (wet), 2,109 (dry) |
| Flow, kgmole/hr | 63.18 |

The syngas may be cooled to 420° C. in a boiler to raise 24.59 kgmole/hr of steam at 42 bar and 399° C. This steam may be returned to the ethanol production plant to supply power or other needs. Sulfur compounds are then removed from the syngas which typically contains 0.0022 kgmole/hr H₂S. This may be done by the use of an iron based chelating agent that converts the sulfur compounds into iron pyrite. Manufacturers of this type of sulfur chelating agent may include Merichem Sulfur-Rite (Houston, Tex.). The syngas may be further cooled to 200° C. for conducting a water gas shift reaction. The moisture content in the shifted syngas may be then knocked out at 40° C. by using direct or indirect condensers to produce approximately 3.40 kg mole/hr water. The shifted cool syngas has the composition noted below in TABLE 20.

TABLE 20
SHIFTED SYNGAS AFTER WATER KNOCK-OUT AT 40° C.

| Composition | Mole % |
|---|---|
| CH₄ | 0.02 |
| CO₂ | 40.84 |
| N₂ | 2.02 |
| CO | 2.29 |
| H₂ | 52.89 |
| H₂O | 1.94 |
| TOTAL | 100.00 |
| Pressure, Bar | 4.00 |
| Temperature, ° C. | 40.00 |
| HHV, BTU/SCF | 179 |
| Kcal/M³ | 1,593 |
| Flow, kgmole/hr | 59.78 |

The shifted syngas may then be compressed to 23 bar. The moisture content in the compressed syngas may be again knocked out at 40° C. by use of direct and/or indirect condensers to produce approximately 0.93 kgmole/hr water.

23.08 kgmole/hr of H₂, which amounts for 73 mole % of the H₂ content in the shifted dry syngas can be recovered at 22 bar and 50° C. having a purity level of 99.999 mole % by a PSA unit along with a low energy fuel gas, at a rate of 35.77 kgmole/hr. The syngas may then be compressed and cooled further before storing and sold as pure H₂.

TABLE 21
H2 CONTENT IN SYNGAS

| Name of Stream | Pure H₂ Produced | Low Energy Fuel Gas |
|---|---|---|
| Comp | Mole % | Mole % |
| CH₄ | — | 0.80 |
| CO₂ | — | 68.23 |
| N₂ | 0.0005 | 3.36 |
| CO | — | 3.54 |
| H₂ | 99.9995 | 23.42 |
| H₂O | — | 0.65 |
| TOTAL | 100.00 | 100.00 |
| Pressure, Bar | 22.00 | 2.00 |
| Temperature, ° C. | 50.00 | 50.00 |
| HHV, BTU/SCF | 325 | 96 |
| Kcal/M³ | 2,892 | 854 |
| Flow, kgmole/hr | 23.08 | 35.77 |

Alternatively, as illustrated in FIG. 11B, the 99.9 mole % of H₂ from the PSA unit may also be sent to an ammonia production process, such the Haber-Bosch process, to convert nitrogen gas and hydrogen gas to anhydrous ammonia, NH₃. The Haber-Bosch process is readily available for sale from Kellogg Brown and Root (Houston, Tex.). The ammonia generated from the ammonia production process may be stored and then sold as, for example, agricultural fertilizer or used for industrial applications. The nitrogen gas feeding into the ammonia production process may originate from an oxygen plant which intakes atmosphere, separates the oxygen from nitrogen, and sends some of the nitrogen gas to be used as feedstock in the ammonia production process. The 90 mole % oxygen stream from the oxygen plant may be used as feedstock in the high pressure gasifier. Unused excess nitrogen gas from the oxygen plant may be processed, compressed, cooled and stored before sale as 99.9 mole % pure N₂.

As another alternative, as illustrated in FIG. 11C, the 99 mole % of H₂ from the PSA unit may be further processed through a dimethyl ether (DME) converter, where the syngas may be converted to methanol, and then to DME. The DME converter may be of a one step or two step process commonly known in industry. The Lurgi MegaDME, by Lurgi GmbH, is one such example of a DME converter. The DME may then be sold, for example, as commercial grade liquid fuel.

Although this example is directed specifically to the production of syngas from deoiled DWG, it is expected that similar results would be obtained by using oil containing or deoiled biomass. Oil containing biomass may be obtained from a biomass harvesting process, and then reduced to appropriate particle size, in a particle size reduction process, before feeding the biomass into a pressurized gasifier to produce syngas. The composition of the syngas, such as the mole % of H₂ and CO, may differ based on the type of biomass used, but the same processes as described herein may be used to produce pure H₂, to convert H₂ into anhydrous ammonia, and/or to convert H₂ into DME.

### Example 12

### Corn-Based Biomass Oil Extraction Comparison of Milled Corn, TS, DWG and DDGS

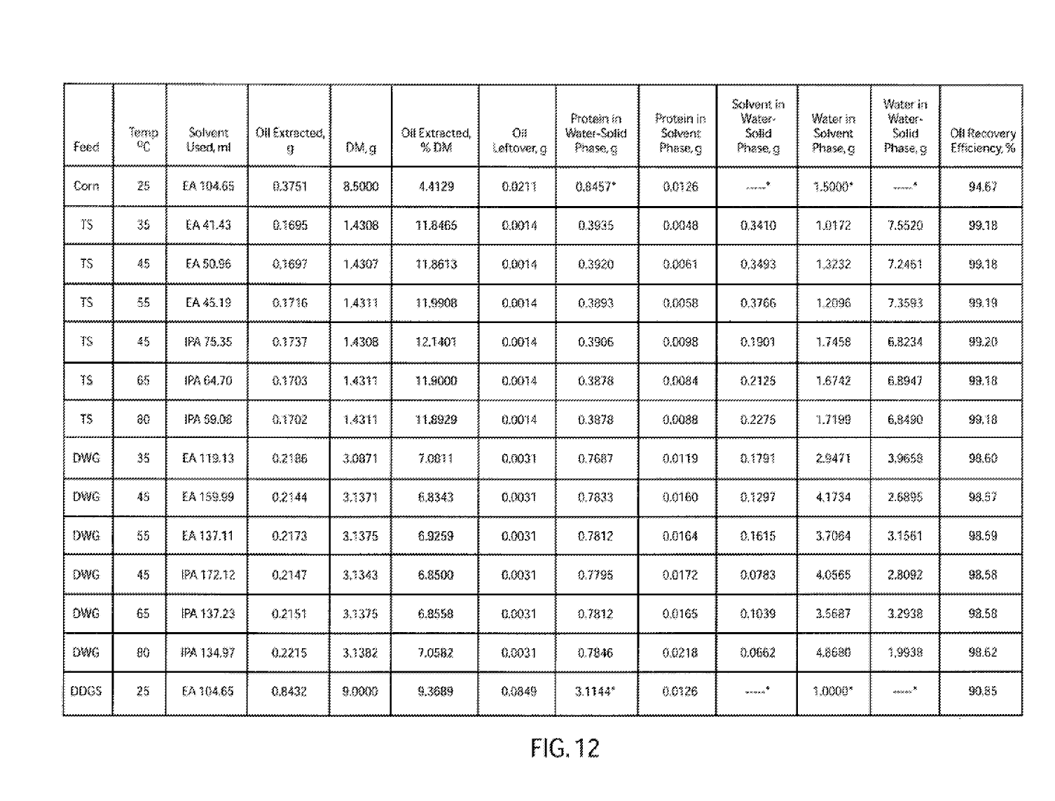

A comparison was performed for the extraction of corn-based biomass oil from milled corn, TS, DWG and DDGS. The comparison includes analysis of each corn-based product and byproduct using oil extraction techniques described herein using ethyl acetate or isopropyl acetate at varying temperatures ranging from 25° C. to 80° C. The results are illustrated in FIG. 12.

As can be seen, using the various techniques described herein, oil can be recovered from milled corn, TS, DWG and DDGS with an efficiency ranging from greater than 90 wt % to greater than 99 wt %, with the greatest recovery percentage available from TS and DWG. These recovery rates are irrespective of the specific alkyl acetate solvent used and temperature applied.

Although the examples provided are directed to milled corn and deoiled biomass, the same methods may be directed to unprocessed corn or unprocessed biomass, and the step of deoiling the biomass may not be necessary.

Although various representative aspects of this invention have been described above with a certain degree of particularity, those skilled in the art could make numerous alterations to the disclosed aspects without departing from the spirit or scope of the inventive subject matter set forth in the specification and claims. Joinder references (e.g., attached, coupled, connected) are to be construed broadly and may include intermediate members between a connection of elements and relative movement between elements. As such, joinder references do not necessarily infer that two elements are directly connected and in fixed relation to each other. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the invention. It may be intended that all matter contained in the above description or shown in the accompanying drawings shall be interpreted as illustrative only and not limiting. Changes in detail or structure may be made without departing from the spirit of the invention as defined in the appended claims.

Although the invention has been described with reference to preferred aspects, persons skilled in the art will recognize that changes may be made in form and detail without departing from the spirit and scope of the invention.

What is claimed:

1. A process for extracting biomass oil from biomass in a process, the process comprising:
obtaining biomass-based product from a biomass-related process;
applying an alkyl acetate solvent to the biomass-based product to extract oil so as to produce an extraction solution of at least biomass-based product solids, oil, solvent and water;
separating the extraction solution into a first phase containing solvent and oil and a second phase containing at least one of water and solids; and
separating the first phase from the second phase and removing the solvent from the oil.

2. The process of claim 1, wherein applying the alkyl acetate solvent comprises applying alkyl acetate solvent prior to fermentation in the process.

3. The process of claim 1, wherein applying the alkyl acetate solvent comprises applying alkyl acetate solvent post fermentation in the process and is applied to at least one byproduct of the fermentation process.

4. The process of claim 3, wherein the alkyl acetate is an azeotrope.

5. The process of claim 1, wherein the removed alkyl acetate solvent is recycled into the process.

6. The process of claim 1, wherein the deoiled second phase comprises a deoiled second phase concentrated by removal of a percentage of water.

7. The process of claim 6, further comprises concentrating the deoiled second phase to produce a filter cake.

8. The process of claim 7, further comprises applying a displacing liquid to the filter cake to dissolve any remaining water and drying any deoiled remaining solids by the application of carbon dioxide.

9. The process of claim 8, wherein the displacing liquid applied to the filter cake is at least one of an:
ethanol azeotrope;
alkyl acetate; and
alkyl actetate azeotrope.

10. The process of claim 8, wherein the displacing liquid carried by the carbon dioxide from the drying step is removed from the carbon dioxide via a condenser and the carbon dioxide is recycled back to the production process.

11. The process of claim 1, wherein the biomass-based product comprises at least one of:
raw corn materials including corn cobs;
milled corn;
raw wheat materials or milled wheat;
raw barley materials or milled barley;
raw rice materials or milled rice;
other raw grains or milled grains;
raw sugarcane materials or milled sugarcane;
raw tree materials;
plant, animal or agricultural waste;
other biological materials;
thick stillage; and
distillers wet grain.

12. The process of claim 1, wherein the biomass-based process comprises an alcohol or biofuel production process that comprises at least one of:
ethanol production; and
biofuel production.

13. The process of claim 1, further comprises producing a byproduct that comprises distillers dried grains with solubles.

14. The process of claim 1, further comprises producing a deoiled biomass-based product that is further processed to produce syngas, the process comprising:
feeding the biomass-based product along with an oxidant, into a pressurized gasifier to produce syngas;
cooling the syngas in a boiler for heat recovery;
removing sulfur compounds from the syngas;
cooling the syngas further for conducting a water gas shift reaction to produce shifted syngas;
removing a percentage of moisture content in the shifted syngas using direct or indirect condensers;
applying compression and further removing a percentage of the moisture content in the shifted syngas using direct or indirect condensers; and
producing a syngas with a higher purity level of hydrogen and separating it from low energy fuel gas in a pressure swing adsorption unit.

15. The process of claim 14, wherein the syngas is further processed to produce at least one of:
anhydrous ammonia;
dimethyl ether;
mixed alcohols;
diesel;
methanol;
butanol; and
hydrogen.

16. A process for producing syngas from oil containing biomass-based product, the process comprising:
obtaining biomass from a biomass harvesting process;
placing the biomass in a particle size reduction process to form a reduced size biomass-based product;
feeding the reduced particle size biomass-based product along with an oxidant, into a pressurized gasifier to produce syngas;
cooling the syngas in a boiler for heat recovery;
removing sulfur compounds from the syngas;
cooling the syngas further for conducting a water gas shift reaction to produce shifted syngas;
removing a percentage of moisture content in the shifted syngas using direct or indirect condensers;
applying compression and further removing a percentage of the moisture content in the shifted syngas using direct or indirect condensers; and
producing a syngas with a higher purity level of hydrogen and separating it from low energy fuel gas in a pressure swing adsorption unit.

17. The process of claim 16, wherein the syngas is further processed to produce at least one of:
anhydrous ammonia;
dimethyl ether;
mixed alcohols;
diesel;
methanol;
butanol; and
hydrogen.

18. A process for production of co-products from an alcohol or biofuel production facility comprising:
placing biomass in a hammermill to produce a milled biomass-based product;
transferring the milled biomass-based product into an extractor and blending the milled biomass-based product with an alkyl acetate solvent mixture to form a combined mixture for extracting an oil;
separating the oil from the deoiled milled biomass-based product;
removing the solvent mixture from the oil and deoiled milled biomass-based product and recycling the solvent mixture back into the extractor; and
transferring the deoiled milled biomass-based product to a fermentor to produce at least one of:
ethanol;
methanol;
butanol;
diesel;
mixed alcohols; and
hydrogen.

19. A process for production of co-products from alcohol or biofuel production comprising:
placing biomass in a particle size reduction process to form a reduced size biomass-based product;
transferring the reduced size biomass-based product into an extractor and blending the biomass-based product with a solvent to form a combined solvent/oil and solvent/solids slurry mixture;
clarifying and separating the solvent/oil from the solvent/solids slurry;
partially removing the solvent from the solvent/oil in a solvent stripper and recycling the solvent back into a solvent blending process;
partially separating the solvent/oil from the solvent/solids slurry mixture in a solvent washing column through the use of a counter-current solvent stream;
further removing the solvent from the solvent/oil and recycling the solvent back into the solvent blending process;
separating a stream in the solvent displacement washing column through the use of a counter-current fluid displacement stream;
removing the displacement fluid from a portion of the stream in a separation process, recycling the displacement fluid back into the solvent displacement washing column, and separating the oil from a portion of the stream in a solvent stripper;
further removing the displacement fluid from a portion of the stream in a separation process, and recycling the displacement fluid back into the solvent displacement washing column; and
transferring a resultant deoiled biomass-based product to a fermentor to produce at least one of:
ethanol;
methanol;
butanol;
diesel;
mixed alcohols; and
hydrogen.

20. A process for production of co-products from an alcohol or biofuel production comprising:
obtaining thick stillage from a fermentation unit; placing the thick stillage in a mixing unit with an alkyl acetate solvent mixture and blending to form a combined mixture for extracting an oil;
allowing the combined mixture to settle into a first phase containing the solvent mixture and oil and a second phase containing deoiled thick stillage and water;
separating the first phase and distilling the first phase to separate the oil from the solvent mixture and recycle the solvent mixture to the mixing unit;
placing the second phase in a reboiler desorption unit to remove unseparated solvent for recycling;
filtering the second phase containing deoiled thick stillage to produce deoiled distillers wet grains and deoiled thin stillage; and
concentrating the deoiled thin stillage to produce a retentate syrup and drying the combined syrup and deoiled distillers wet grains to produce oil free distillers dry grains with solubles.

21. A process for production of co-products from an alcohol or biofuel production comprising:
obtaining distillers wet grain from a post fermentation process;
blending the distillers wet grain with an alkyl acetate solvent mixture to form a combined mixture for extracting an oil;
allowing the combined mixture to settle into a first phase containing the solvent mixture and oil and a second phase containing deoiled distillers wet grain and water;
separating the first phase and distilling the first phase to separate oil from the solvent mixture and recycle the solvent mixture;
placing the second phase into a reboiler desorption unit to remove unseparated solvent for recycling to produce a deoiled, dewatered second phase;
removing additional water content from the deoiled, dewatered second phase; and
placing the deoiled, dewatered second phase into a combustion/boiler unit for the production of energy.

22. A process for production of co-products from an alcohol or biofuel production comprising:
obtaining distillers dry grains with solubles from a post fermentation process;
blending the distillers dry grains with solubles with an alkyl acetate solvent mixture to form a combined mixture for extracting an oil;
separating the oil to produce deoiled dry grains and recycling the solvent mixture;
removing the solvent remaining in the deoiled dry grains with solubles to produce oil free distillers dry grains with solubles; and
recycling the remaining solvent.

23. A process for drying a wet solid co-product containing an amount of moisture from an alcohol or biofuel production process comprising:
obtaining an ethanol stream from the production process, or an alkyl acetate stream;
converting the ethanol or alkyl acetate into an azeotrope;
applying the azeotrope to a wet solid co-product to remove moisture and form an ethanol or alkyl acetate rich solid co-product;
applying a stream of hot carbon dioxide gas from the alcohol or biofuel production process to the ethanol or alkyl acetate rich solid co-product so as to remove ethanol or alkyl acetate from the solid co-product and form an ethanol or alkyl acetate-carbon dioxide stream;
condensing the ethanol or alkyl acetate from the ethanol or alkyl acetate/carbon dioxide stream; and
recycling the carbon dioxide back into the production process, thereby leaving a dried solid co-product.

24. A system for removing oil from biomass-based products prior to fermentation in an alcohol or biofuel production comprising:
a hammermill configured to reduce whole or raw biomass to a milled biomass-based product and produce an output stream of milled biomass-based product;
an extractor containing a solvent and in communication with the output stream from the hammermill configured to blend the milled biomass-based product with the solvent to form a solvent mixture and produce a solvent mixture stream; and
a solvent stripper in operable communication with an output solvent mixture stream from the extractor, the solvent stripper configured to remove solvent from the solvent mixture to form a deoiled milled biomass-based product stream, the solvent stripper further being in operable communication with the extractor to provide at least a portion of the solvent stream to the extractor and in operable communication with a fermentor to provide a deoiled milled biomass-based product stream to the fermentor for the production of at least one of:
ethanol;
mixed alcohols;
diesel;
methanol;
butanol; and
hydrogen.

25. A system for removing oil from a biomass-based product post fermentation in an alcohol or biofuel production comprising:
an extractor containing a solvent and in operable communication with a fermentor, wherein the extractor is configured to receive an output thick stillage stream from the fermentor and form a solvent mixture;
a phase settler in operable communication with the extractor configured to receive the solvent mixture, the phase settler configured to separate a deoiled biomass-based product phase from an oil in solvent phase;
a distillation assembly in operable communication with the phase settler configured to receive an oil in solvent phase stream, the distillation assembly configured to separate the oil from the solvent, wherein the distillation assembly is in operable communication with the extractor to provide at least a portion of the solvent to the extractor; and
a reboiler desorption unit operably attached to the phase settler configured to receive a deoiled biomass-based product phase stream, the reboiler desorption unit configured to receive solvent remaining in the deoiled biomass-based product phase, the reboiler desorption unit in operable communication with the extractor configured to provide at least a portion of the solvent stream to the extractor.

26. The system of claim 25, further comprising:
a filtration device operably attached to the reboiler desorption unit configured to receive the deoiled biomass-based product stream, the filtration device configured to separate the deoiled thick stillage into an oil free distillers wet grain;
a membrane configured for water removal in communication with an oil free thin stillage; and
a drying device to produce an oil free distillers dried grain with solubles.

27. A system for removing oil from a biomass-based product post fermentation in an alcohol or biofuel production comprising:
an extractor containing a solvent and in operable communication with a fermentor, wherein the extractor is configured to receive an output distillers wet grain stream from centrifuging of a thick stillage from the fermentor and forming a solvent mixture;
a phase settler in operable communication with the extractor configured to receive the solvent mixture, the phase settler configured to separate a deoiled biomass-based product phase from an oil in solvent phase;
a distillation assembly in operable communication with the phase settler configured to receive an oil in solvent phase stream, the distillation assembly configured to separate the oil from the solvent, wherein the distillation assembly is in operable communication with the extractor configured to provide at least a portion of the solvent to the extractor; and
a reboiler desorption unit operably attached to the phase settler configured to receive a deoiled biomass-based product phase stream, the reboiler desorption unit configured to remove solvent remaining in the deoiled biomass-based product phase, the reboiler desorption unit in operable communication with the extractor configured to provide at least a portion of the solvent stream to the extractor.

28. The system of claim 27, further comprising a water removal device in operable communication with the reboiler desorption unit configured to receive the deoiled biomass-based product stream, the water removal device configured to remove of a percentage of water from the deoiled biomass-based product stream.

29. The system of claim 28, further comprising a combustion device in operable communication with the water removal device configured to receive deoiled distillers wet grain biomass-based product stream, the combustion device configured to generate energy output from the deoiled biomass-based product stream.

30. The system of claim 28, wherein an optimum moisture level in the deoiled biomass-based product stream for combustion is obtained and which comprises approximately 30 wt % moisture.

31. A system for removing oil from a biomass-based product post fermentation in an alcohol or biofuel production comprising:
an extractor containing a solvent and in operable communication with a fermentor, wherein the extractor is configured to receive an output distillers dried grains with solubles stream from the production facility and form a solvent mixture;
a phase settler in operable communication with the extractor configured to receive the solvent mixture, the phase settler configured to separate a deoiled biomass-based product phase from an oil in solvent phase;
a distillation assembly in operable communication with the phase settler configured to receive an oil in solvent phase stream, the distillation assembly configured to separate the oil from the solvent, wherein the distillation assembly is in operable communication with the extractor configured to provide at least a portion of the solvent to the extractor; and
a reboiler desorption unit operably attached to the phase settler configured to receive a deoiled biomass-based product phase stream, the reboiler desorption unit configured to remove solvent remaining in the deoiled biomass-based product phase, the reboiler desorption unit in operable communication with the extractor configured to provide at least a portion of the solvent stream to the extractor.
